(19) United States
(12) Patent Application Publication
(10) Pub. No.: US 2026/0015483 A1
(43) Pub. Date: Jan. 15, 2026

(54) DEPOLYMERIZATION METHOD FOR RESIN HAVING FLUORENE SKELETON, AND PRODUCT AND APPLICATION THEREOF

(71) Applicant: Osaka Gas Chemicals Co., Ltd., Osaka-shi, Osaka (JP)

(72) Inventor: Hiroshi YOSHIMURA, Osaka (JP)

(73) Assignee: Osaka Gas Chemicals Co., Ltd., Osaka-shi, Osaka (JP)

(21) Appl. No.: 18/993,732

(22) PCT Filed: Jul. 11, 2023
(86) PCT No.: PCT/JP2023/025537

§ 371 Date: Jan. 13, 2025

(30) Foreign Application Priority Data
Jul. 15, 2022 (JP) 2022-113772
Mar. 31, 2023 (JP) 2023-057824

(51) Int. Cl. C08J 11/26; C07C 41/26; C07C 41/40; C07C 67/03; C07C 67/52; C07C 68/065; C08G 63/91; C08J 11/16; C08J 11/24

(52) U.S. Cl. CPC C08J 11/26; C07C 41/26; C07C 41/40; C07C 67/03; C07C 67/52; C07C 68/065; C08G 63/916; C08J 11/16; C08J 11/24; C08J 2367/06

(57) ABSTRACT

A fluorene-containing resin with a fluorene skeleton and an ester bond and/or a carbonate ester bond is depolymerized by a depolymerization method that includes allowing the fluorene-containing resin to react with a carbonate ester in the presence of a hydrolysis catalyst to decompose the fluorene-containing resin, obtaining a decomposition product. This depolymerization method allows simple or easy depolymerization of the fluorene-containing resin. The decomposition product may contain a monocarbonate ester form of a diol and/or a dicarbonate ester form of a diol. In the depolymerization method, the decomposition product may be allowed to react with an alcohol to obtain a diol.

### TECHNICAL FIELD

The present disclosure relates to a depolymerization of a resin with a fluorene skeleton and an ester bond and/or a carbonate ester bond,

### BACKGROUND ART

In recent years, immediate realization of decarbonized society has been awaited on a global scale, and SDGs (Sustainable Development Goals) are set as specific goals. As a part thereof, recycling of used plastics is also being promoted. In particular, a polyester-series resin, typified by a poly (ethylene terephthalate) (PET), is distributed in large quantities as beverage bottles and textile products, and recycling thereof is intended greatly, A known example of a method for recycling a plastic is a chemical recycling which includes chemically decomposing a plastic into a low molecular weight compound and producing a plastic from the resulting low molecular weight compound. The chemical recycling is being noticed from the viewpoint of a high quality of the recycled product and a repeated use of the recycled product.

As a chemical recycling method, Japanese Patent Application Laid-Open Publication Nos. 2003-128600 (JP 2003-128600 A, Patent Document 1) and 2003-119316 (JP 2003-119316 A, Patent Document 2) disclose a method for decomposing a PET by addition of an excess amount of ethylene glycol to produce terephthalic acid.

WO 2004/041917 (Patent Document 3) discloses a method for decomposing a polyester into a monomer or an oligomer by hydrolysis using subcritical water or supercritical water.

Green Chem, 2021, 23, 9412-9416 (Nonpatent Document 1) discloses that methanol, dimethyl carbonate (DMT), and lithium methoxide are mixed with a flaky PET at an appropriate ratio and the mixture is allowed to react at 28° C. for 5 hours to produce dimethyl terephthalate at a yield of 74% and that the PET is completely decomposed in 5 hours when the reaction temperature is changed to 50° C. This method produces dimethyl terephthalate and ethylene carbonate by decomposition reaction. This document states that the decomposition reaction proceeds due to functioning dimethyl carbonate as an ethylene glycol scavenger and due to the stable 5-membered ring structure of ethylene carbonate produced by the reaction.

### CITATION LIST

### Patent Literature

[Patent Document 1] JP 2003-128600 A
[Patent Document 2] JP 2003-119316 A
[Patent Document 3] WO 2004/041917

### NONPATENT LITERATURE

[Nonpatent Document 1] Shinji Tanaka et al., “Capturing ethylene glycol with dimethyl carbonate towards depolymerisation of polyethylene terephthalate at ambient temperature”, Green Chem, 2021, 23, 9412-9416

### SUMMARY OF INVENTION

### Technical Problem

However, the depolymerization methods in Patent Documents 1 to 3 include a reaction under high-temperature and high-pressure conditions. For example, all the methods use a reaction temperature of not lower than 200° C. and fail to easily produce a monomer, resulting in a low productivity.

Meanwhile, the depolymerization method in Nonpatent Document 1 enables depolymerization of the PET at a low temperature. However, the document fails to describe a polyester-series resin other than the PET. For example, a fluorene skeleton-containing polyester-series resin (or a polyester-series resin with a fluorene skeleton) has recently been used in optical and other applications. The fluorene skeleton-containing polyester-series resin is quite different in structure from a widely used polyester such as a PET due to the bulkiness of the fluorene skeleton, and exhibits a different chemical behavior along with the specificity of the fluorene skeleton. However, Nonpatent Document 1 fails to describe a fluorene skeleton-containing polyester-series resin.

It is therefore an object of the present disclosure to provide a method for simply or easily depolymerizing a resin with (or having) a fluorene skeleton and an ester bond and/or a carbonate ester bond in a molecule thereof and an application thereof.

Another object of the present disclosure is to provide a process for producing a novel carbonate ester compound by depolymerization of a resin with a fluorene skeleton and an ester bond and/or a carbonate ester bond.

### Solution to Problem

The inventor of the present invention made intensive studies to achieve the above objects and finally found that a reaction of a resin having a fluorene skeleton and an ester bond and/or a carbonate ester bond with a carbonate ester in the presence of a hydrolysis catalyst simply or easily enables decomposition (depolymerization) of the resin. The present disclosure was accomplished based on the above findings.

That is, an aspect of the present disclosure (an aspect [1]) is a depolymerization method for depolymerizing a fluorene-containing resin, the fluorene-containing resin having a fluorene skeleton and an ester bond and/or a carbonate ester bond (hereinafter, the resin may simply be referred to as a “fluorene-containing resin”), the method including allowing the fluorene-containing resin to react with a carbonate ester in the presence of a hydrolysis catalyst to obtain or provide a decomposition product.

An aspect of the present disclosure may be an aspect (an aspect [2]) in which the decomposition product in the aspect [1] contains a monocarbonate ester form of a diol and/or a dicarbonate ester form of a diol, or may be an aspect (an aspect [3]) in which the decomposition product in the aspect [2] further contains a dicarboxylic acid and/or an ester thereof.

An aspect of the present disclosure may be an aspect (an aspect [4]) in which any one of the aspects [1] to [3] further comprises allowing the decomposition product to react with an alcohol to obtain a diol.

An aspect of the present disclosure may be an aspect (an aspect [5]) in which the fluorene-containing resin in any one of the aspects [1] to [4] is a polyester-series resin containing, as a polymerization component, a dicarboxylic acid component represented by the following formula (1):

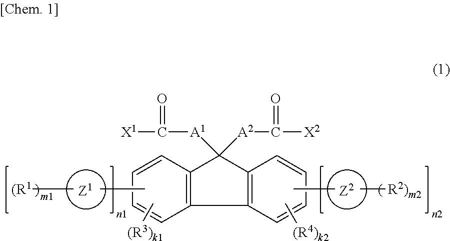

wherein
a ring Z¹ and a ring Z² independently represent an arene ring,
R¹ and R² independently represent a substituent, m1 and m2 independently denote an integer of not less than 0,
n1 and n2 independently denote an integer of 0 to 4,
A¹ and A² independently represent an alkylene group,
X¹ and X² independently represent a hydroxyl group, an alkoxy group, or a halogen atom,
R³ and R⁴ independently represent a substituent, and k1 and k2 independently denote an integer of 0 to 4 and/or
a diol represented by the following formula (2):

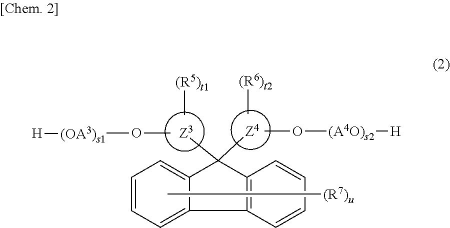

wherein
a ring Z³ and a ring Z⁴ independently represent an arene ring,
A³ and A⁴ independently represent an alkylene group, s1 and s2 independently denote an integer of not less than 0,
R⁵ and R² independently represent a substituent, t1 and t2 independently denote an integer of not less than 0,
R⁷ represents a substituent, and u denotes an integer of 0 to 8.

An aspect of the present disclosure may be an aspect (an aspect [6]) in which the fluorene-containing resin in the aspect [5] is a polyester-series resin containing the dicarboxylic acid component represented by the formula (1) as the polymerization component, and in the formula (1), n1 and n2 each denote 0, or n1 and n2 each denote 1 and the ring Z³ and the ring Z⁴ independently represent a condensed polycyclic arene ring; or may be an aspect (an aspect [7]) in which the fluorene-containing resin in the aspect [5] or [6] is a polyester-series resin containing the diol represented by the formula (2) as the polymerization component.

An aspect of the present disclosure may be an aspect (an aspect [8]) in which the fluorene-containing resin in any one of the aspects [1] to [4] is a polycarbonate ester-series resin containing, as a polymerization component, a diol represented by the following formula (2):

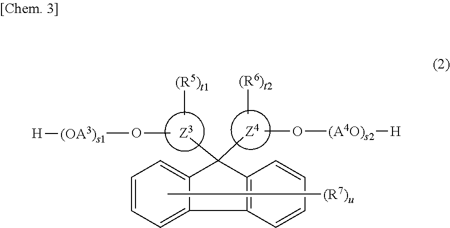

wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

An aspect of the present disclosure may be an aspect (an aspect [9]) in which the fluorene-containing resin in any one of the aspects [1] to [8] contains, as a polymerization component, a diol represented by the following formula (5):

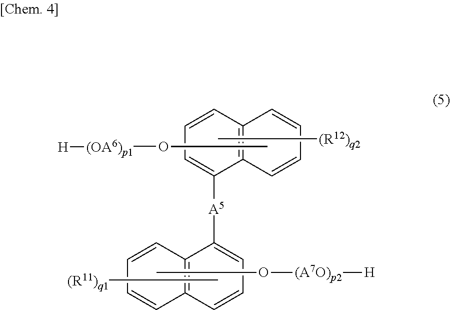

wherein
A⁵ represents a direct bond (a single bond) or an alkylene group,
A⁶ and A⁷ independently represent an alkylene group, p1 and p2 independently denote an integer of not less than 0,
R¹¹ and R¹² independently represent a substituent, and q1 and q2 independently denote an integer of 0 to 6.

An aspect (an aspect [10]) of the present disclosure is a process for producing a fluorene skeleton-containing dicarboxylic acid (or a dicarboxylic acid with a fluorene skeleton) and/or an ester thereof, the process including allowing a polyester-series resin containing a fluorene skeleton-containing dicarboxylic acid component (a fluorene-containing dicarboxylic acid component) as a polymerization component to react with a carbonate ester in the presence of a hydrolysis catalyst to decompose the polyester-series resin.

An aspect (an aspect [11]) of the present disclosure is a process for producing a fluorene skeleton-containing diol (or a diol with a fluorene skeleton), the process including
a first decomposition step of allowing a polyester-series resin containing a fluorene skeleton-containing diol (a fluorene-containing diol) as a polymerization component to react with a carbonate ester in the presence of a hydrolysis catalyst to decompose the polyester-series resin, obtaining a first decomposition product containing a dicarboxylic acid and/or an ester thereof, and a monocarbonate ester form of a diol and/or a dicarbonate ester form of a diol, and
a second decomposition step of allowing the first decomposition product to react with an alcohol to obtain a diol.

An aspect (an aspect [12]) of the present disclosure is a method for recovering a fluorene skeleton-containing monomer component (or a monomer component with a fluorene skeleton), the method including
a first decomposition step of allowing a polyester-series resin with a fluorene skeleton and an ester bond to react with a carbonate ester in the presence of a hydrolysis catalyst to decompose the polyester-series resin, obtaining a decomposition product containing a dicarboxylic acid and/or an ester thereof, and a monocarbonate ester form of a diol and/or a dicarbonate ester form of a diol, and
a second decomposition step of allowing the decomposition product to react with an alcohol to obtain a diol.

An aspect (an aspect [13]) of the present disclosure is a method for recovering a fluorene skeleton-containing monomer component, the method including
a first decomposition step of allowing a polycarbonate ester-series resin with a fluorene skeleton and a carbonate ester bond to react with a carbonate ester in the presence of a hydrolysis catalyst to decompose the polycarbonate ester-series resin, obtaining a first decomposition product containing a monocarbonate ester form of a diol and/or a dicarbonate ester form of a diol, and
a second decomposition step of allowing the first decomposition product to react with an alcohol to obtain a diol.

An aspect (an aspect [14]) of the present disclosure is a monocarbonate ester form of a diol represented by the following formula (3);

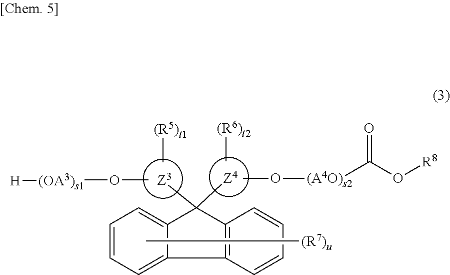

wherein
a ring Z³ and a ring Z⁴ independently represent an arene ring,
A³ and A⁴ independently represent an alkylene group, s1 and s2 independently denote an integer of not less than 0,
R⁸ represents a hydrocarbon group,
R⁵ and R⁶ independently represent a substituent, t1 and t2 independently denote an integer of not less than 0,
R⁷ represents a substituent, and u denotes an integer of 0 to 8.

An aspect (an aspect [15]) of the present disclosure is a dicarbonate ester form of a diol represented by the following formula (4):

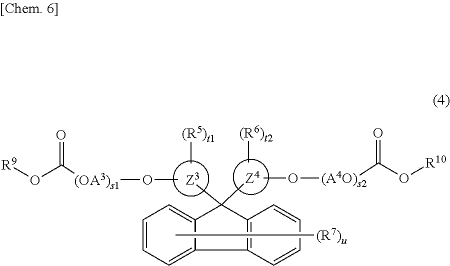

wherein
a ring Z³ and a ring Z⁴ independently represent an arene ring,
A³ and A⁴ independently represent an alkylene group, s1 and s2 independently denote an integer of not less than 0,
R⁹ and R¹⁰ independently represent a hydrocarbon group,
R⁵ and R⁶ independently represent a substituent, t1 and t2 independently denote an integer of not less than 0,
R⁷ represents a substituent, and u denotes an integer of 0 to 8.

An aspect (an aspect [16]) of the present disclosure is a process for producing a monocarbonate ester form of a diol recited in the aspect [14], the process including allowing a fluorene-containing resin with a fluorene skeleton and an ester bond and/or a carbonate ester bond to react with a carbonate ester in the presence of a hydrolysis catalyst to decompose the fluorene-containing resin, wherein
the fluorene-containing resin is a fluorene-containing resin, as a polymerization component, a diol represented by the following formula (2):

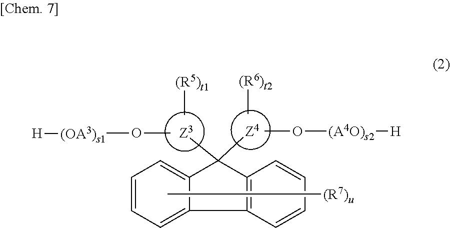

wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

An aspect (an aspect [17]) of the present disclosure is a process for producing a dicarbonate ester form of a diol recited in the aspect [15], the process including allowing a fluorene-containing resin with a fluorene skeleton and an ester bond and/or a carbonate ester bond to react with a carbonate ester in the presence of a hydrolysis catalyst to decompose the fluorene-containing resin, wherein
the fluorene-containing resin is a fluorene-containing resin containing, as a polymerization component, a diol represented by the following formula (2):

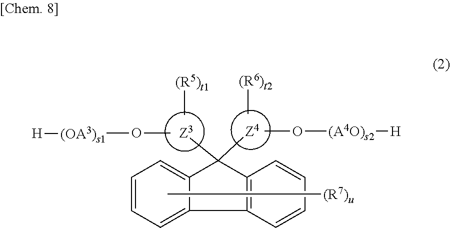

wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

An aspect (an aspect [18]) of the present disclosure is a method for recycling a fluorene-containing resin with a fluorene skeleton and an ester bond and/or a carbonate ester bond, the method including
a depolymerization step of allowing the fluorene-containing resin to react with a carbonate ester in the presence of a hydrolysis catalyst to obtain a decomposition product, and
a polymerization step of polymerizing the decomposition product obtained in the depolymerization step to obtain a newly produced fluorene-containing resin.

An aspect (an aspect [19]) of the present disclosure may be an aspect in which a monomeric raw material for the newly produced fluorene-containing resin is supplemented in the polymerization step in the aspect [18].

In the description and the claims of this application, the number of carbon atoms in a substituent or others may be represented as C₁, C₆, C₁₀, or others. For example, “C₁alkyl group” means an alkyl group having one carbon atom, and “C₆₋₁₀aryl group” means an aryl group having 6 to 10 carbon atoms.

### Advantageous Effects of Invention

The depolymerization method for the fluorene-containing resin of the present disclosure enables simple or easy depolymerization of a resin with a fluorene skeleton in the molecule and with an ester bond and/or a carbonate ester bond in the molecule, for example, a polyester-series resin or a polycarbonate ester-series resin.

### BRIEF DESCRIPTION OF DRAWINGS

FIG. 1 is a chart showing a molecular weight distribution of a polyester before depolymerization obtained in Comparative Example 1.

FIG. 2 is a chart showing a molecular weight distribution of a decomposition product resulting from depolymerization of a polyester obtained in Comparative Example 1.

FIG. 3 is a chart showing a molecular weight distribution of a polyester before depolymerization obtained in Comparative Example 2.

FIG. 4 is a chart showing a molecular weight distribution of a decomposition product resulting from depolymerization of a polyester obtained in Comparative Example 2.

### DESCRIPTION OF EMBODIMENTS

### [Fluorene-Containing Resin]

A resin to be depolymerized in the present disclosure is a fluorene-containing resin with (or having) a fluorene skeleton and an ester bond and/or a carbonate ester bond in a molecule thereof [or a (carbonate) ester bond-containing resin]. Moreover, in the description and the claims of this application, the (carbonate) ester bond means an ester bond and/or a carbonate ester bond.

The resin with a (carbonate) ester bond in a molecule thereof may include, for example, a polyester, a polyester-polycarbonate ester (a polyester-carbonate ester), and a polycarbonate ester.

The fluorene skeleton may be in either main chain or side chain of the fluorene-containing resin. Moreover, the fluorene skeleton is linked to the (carbonate) ester bond directly from the fluorene skeleton or through a divalent connecting group. The (carbonate) ester bond or the divalent connecting group may be bonded at any position of the fluorene skeleton without particular limitation, and is preferably bonded at 9, 9-positions or 2, 7-positions of the fluorene ring, particularly preferably 9, 9-positions of the fluorene ring. The divalent connecting group may be a connecting group at least containing a hydrocarbon group,

Among the fluorene-containing resins, preferred is a fluorene-containing resin containing at least an ester bond formed by polymerization of at least a dicarboxylic acid component and a diol component, or a fluorene-containing resin at least containing a carbonate ester bond formed by polymerization of at least a diol component. Aa such a fluorene-containing resin, preferred is a fluorene-containing polyester-series resin typified by a fluorene-containing polyester resin and a fluorene-containing polyester-carbonate ester resin, or a fluorene-containing polycarbonate ester-series resin typified by a fluorene-containing polycarbonate ester resin; further preferred is a fluorene-containing polyester-series resin or a fluorene-containing polycarbonate ester resin; and particularly preferred is a fluorene-containing polyester resin.

### [Fluorene-Containing Polyester-Series Resin]

The fluorene-containing polyester-series resin contains a dicarboxylic acid component and a diol component as polymerization components, and at least one of the dicarboxylic acid component and the diol component contains a fluorene skeleton-containing component.

### (Fluorene-Containing Dicarboxylic Acid Component)

In a case where the dicarboxylic acid component as a polymerization component contains a fluorene skeleton-containing dicarboxylic acid component (a dicarboxylic acid component with a fluorene skeleton or a fluorene-containing dicarboxylic acid component), the fluorene-containing dicarboxylic acid component is not particularly limited to a specific one, and is preferably a dicarboxylic acid component represented by the above-mentioned formula (1).

In the formula (1), examples of the arene rings (or aromatic hydrocarbon rings) represented by the ring Z³ and the ring Z⁴ may include a monocyclic arene ring such as a benzene ring, and a polycyclic arene ring; examples of the polycyclic arene ring may include a condensed polycyclic arene ring (a condensed polycyclic aromatic hydrocarbon ring) and a ring-assemblies (or ring-aggregated) arene ring (a ring-assemblies polycyclic aromatic hydrocarbon ring).

Examples of the condensed polycyclic arene ring may include a condensed bicyclic arene ring, specifically a condensed bicyclic C₁₀₋₁₆arene ring such as a naphthalene ring and an indene ring; and a condensed bi-to tetra-cyclic arene ring such as a condensed tricyclic arene ring. Examples of the condensed tricyclic arene ring may include a condensed tricyclic C₁₄₋₂₀arene ring such as an anthracene ring and a phenanthrene ring. The preferred condensed polycyclic arene ring is a condensed bicyclic C₁₀₋₁₄arene ring such as a naphthalene ring, Examples of the ring-assemblies arene ring may include a biarene ring such as a biphenyl ring, a phenylnaphthalene ring, and a binaphthyl ring; and a terarene ring such as a terphenyl ring. The preferred ring-assemblies arene ring is a C₁₂₋₁₈biarene ring such as a biphenyl ring.

The preferred rings Z¹ and Z² are each a C₆₋₁₄arene ring, preferably a C₆₋₁₂arene ring such as a benzene ring, a naphthalene ring, and a biphenyl ring, further preferably a C₆₋₁₀arene ring such as a benzene ring and a naphthalene ring, and most preferably a naphthalene ring.

The species of the rings Z¹ and Z² may be different from each other and are usually the same.

Moreover, each of the ring Z¹ and the ring Z² may be substituted at any of 1- to 4-positions and 5-to 8-positions of the fluorene ring, and may usually be substituted at 2-position, 3-position, and/or 7-position, 8-position. The preferred substitution positions (or bonding positions) of the rings Z¹ and Z² are symmetrical positions on the paper surface in the formula (1), such as 1, 8-positions, 2,7-positions, 3,6-positions, and 4,5-positions of the fluorene ring, and particularly 2, 7-positions. In a case where each of the rings Z¹ and Z² is a naphthalene ring, either 1-position or 2-position of the naphthalene ring may be bonded to the of the fluorene ring. From the viewpoint of improving heat resistance, 1-position of the naphthalene ring is particularly preferably bonded to the fluorene ring. From the viewpoint of preparing a resin having a high refractive index, a low Abbe's number, and a low birefringence (or a large negative birefringence) in a well-balanced manner, 2-position of the naphthalene ring is particularly preferably bonded to the fluorene ring.

Examples of the substituents represented by R¹ and R² may include a halogen atom, an alkyl group, an aryl group, an alkoxy group, an acyl group, a nitro group, a cyano group, and a mono- or di-substituted amino group.

The halogen atom may include a fluorine atom, a chlorine atom, a bromine atom, and an iodine atom,

The alkyl group may be a straight- or branched-chain alkyl group, and may include a C₁₋₁₀alkyl group such as methyl group, ethyl group, propyl group, isopropyl group, n-butyl group, and t-butyl group, preferably a C₁₋₆alkyl group, and further preferably a C₁₋₄alkyl group. Examples of the aryl group may include a C₆₋₁₂aryl group such as phenyl group, an alkylphenyl group, biphenylyl group, and naphthyl group; and a mono-to tri-C₁₋₄alkyl-phenyl group such as methylphenyl group (or tolyl group) and dimethylphenyl group (or xylyl group).

Examples of the alkoxy group may include a C₁₋₁₀alkoxy group such as methoxy group, ethoxy group, propoxy group, n-butoxy group, and t-butoxy group.

Examples of the acyl group may include a C₁₋₆alkyl-carbonyl group such as acetyl group.

Examples of the mono- or di-substituted amino group may include a mono- or di-C₁₋₄alkylamino group such as dimethylamino group; and a bis(C₁₋₄alkyl-carbonyl) amino group such as diacetylamino group.

Representative examples of the groups R¹ and R² may include the alkyl group, the aryl group, the alkoxy group, the acyl group, the nitro group, and the cyano group. The preferred groups R¹ and R² are each the alkyl group, specifically a C₁₋₆alkyl group such as methyl group; the alkoxy group, specifically a C₁₋₄alkoxy group such as methoxy group; and particularly preferably a C₁₋₄alkyl group such as methyl group. In a case where each of the groups R¹ and R² is the aryl group, the groups R¹ and R² may form the ring-assemblies arene rings together with the rings Z¹ and Z², respectively.

Each of the substitution numbers (or the numbers of substituents) m1 and m2 is an integer of 0 to 4, preferably an integer of 0 to 2, further preferably 0 or 1, and more preferably 0. In a case where each of m1 and m2 is an integer of not less than 2, the species of the two or more groups R¹ and R² may be the same or different from each other.

Each of the substitution numbers n1 and n2 may be selected from a range of an integer of 0 to 4, and is, for example, an integer of 0 to 3, preferably an integer of 0 to 2, further preferably 0 or 1, and more preferably 1. Use of a compound in which each of n1 and n2 is not less than 1 allows the fluorene-containing polyester-series resin to have a higher refractive index, a higher glass transition temperature, a lower Abbe's number, and a lower absolute value of birefringence.

Examples of the alkylene groups represented by the groups A¹ and A² may include a C₁₋₈alkylene group such as methylene group, ethylene group, trimethylene group, propylene group, 1,4-butanediyl group, and 2-methylpropane-1,3-diyl group. Among them, a C₁₋₆alkylene group is preferred, a C₂₋₄alkylene group is further preferred, a C₂₋₃alkylene group such as ethylene group and propylene group is more preferred, and ethylene group is most preferred.

As the alkoxy groups represented by X¹ and X², there may be mentioned, for example, a C₁₋₄alkoxy group such as methoxy group, ethoxy group, propoxy group, and t-butoxy group, and a C₁₋₂alkoxy group is preferred. Examples of the halogen atom may include a chlorine atom and a bromine atom. The preferred X¹ and X² are each hydroxyl group, methoxy group, and ethoxy group. In order to react at a low temperature, the halogen atom such as the chlorine atom is also preferred. Among them, the C₁₋₂alkoxy group such as methoxy group and ethoxy group is further preferred, and methoxy group is most preferred.

Examples of the substituents represented by R³ and R⁴ may include an alkyl group, a halogen atom such as a fluorine atom, a chlorine atom, and a bromine atom, and a cyano group; examples of the alkyl group may include a C₁₋₆alkyl group such as methyl group, ethyl group, and t-butyl group. The preferred R³ and R⁴ are each a C₁₋₄alkyl group such as methyl group.

The substitution positions of R³ and R⁴ may be 1-position, 2-position, 7-position, 3, 6-positions, 4, 5-positions, or 2, 7-positions of the fluorene ring. Each of the substitution number k1 of R³ and the substitution number k2 of R⁴ may be selected from integers 0 to 4, and is, for example, an integer of 0 to 3, preferably an integer of 0 to 2, further preferably 0 or 1, and more preferably 0. In a case where each of k1 and k2 is an integer of not less than 2, the species of the two or more substituents R³ and R⁴ may be the same or different from each other.

Representative examples of the fluorene-containing dicarboxylic acid component represented by the formula (1) may include a dicarboxylic acid component in which each of n1 and n2 is 0, that is, a 9, 9-bis(carboxyalkyl) fluorene; and a dicarboxylic acid component in which each of n1 and n2 is 1, that is, a 9, 9-bis(carboxyalkyl)-diarylfluorene,

Specifically, the preferred fluorene-containing dicarboxylic acid component may be a dicarboxylic acid component represented by the following formula (1a), (1b), or (1c):

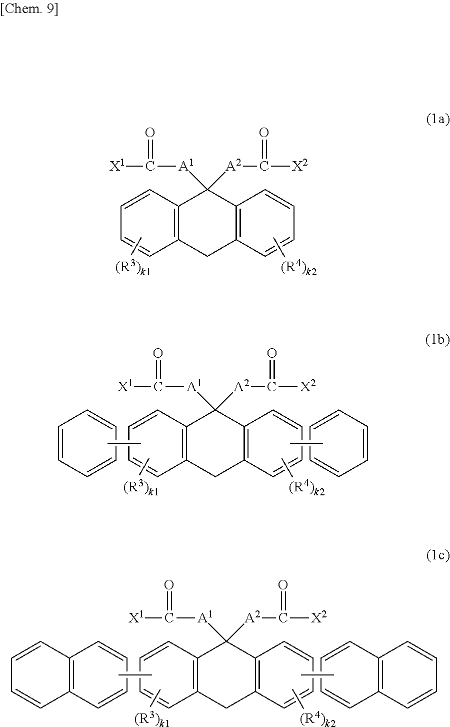

wherein A¹, A², X¹, X², R³, R⁴, k1, and k2 each have the same meanings as defined in the formula (1),

The compound of the formula (1) in which each of n1 and n2 is 0 [a 9, 9-bis(carboxyalkyl) fluorene corresponding to the formula (1a)] may include, for example, a 9, 9-bis(carboxy-C₂₋₆alkyl) fluorene such as 9, 9-bis(2-carboxyethyl) fluorene and 9, 9-bis(2-carboxypropyl) fluorene, and is preferably a 9,9-bis(carboxy-C₂₋₄alkyl) fluorene, further preferably a 9, 9-bis(carboxy-C₂₋₃alkyl) fluorene, more preferably 9, 9-bis(2-carboxyethyl) fluorene and 9, 9-bis(2-carboxypropyl) fluorene, and most preferably 9, 9-bis(2-carboxyethyl) fluorene. Such a compound may be an ester-forming derivative, and is preferably a C₁₋₄alkyl ester such as a methyl ester and an ethyl ester and most preferably a C₁₋₄alkyl ester such as a methyl ester.

In the description and the claims of this application, the ester-forming derivative is also meant to include a halocarboxylic acid such as an acyl chloride, an alkyl ester such as a methyl ester, and an acid anhydride,

Examples of the compound of the formula (1) in which each of n1 and n2 is 1 (the 9,9-bis(carboxyalkyl)-diarylfluorene) may include a 9, 9-bis(carboxyalkyl)-diphenylfluorene corresponding to the formula (1b), specifically a 9, 9-bis(carboxy-C₂₋₆alkyl)-diphenylfluorene such as 9, 9-bis(2-carboxyethyl)-1, 8-diphenylfluorene, 9, 9-bis(2-carboxyethyl)-2,7-diphenylfluorene, 9, 9-bis(2-carboxyethyl)-3, 6-diphenylfluorene, 9, 9-bis(2-carboxyethyl)-4,5-diphenylfluorene, and 9, 9-bis(2-carboxypropyl)-2,7-diphenylfluorene; and a 9, 9-bis(carboxyalkyl)-dinaphthylfluorene corresponding to the formula (1c), specifically a 9, 9-bis(carboxy-C₂-6alkyl)-dinaphthylfluorene such as 9, 9-bis(2-carboxyethyl)-1, 8-di(2-naphthyl) fluorene, 9, 9-bis(2-carboxyethyl)-2,7-di(2-naphthyl) fluorene, 9, 9-bis(2-carboxyethyl)-3, 6-di(2-naphthyl) fluorene, 9, 9-bis(2-carboxyethyl)-4,5-di(2-naphthyl) fluorene, 9, 9-bis(2-carboxypropyl)-2,7-di(2-naphthyl) fluorene, and 9, 9-bis(2-carboxyethyl)-2,7-di(1-naphthyl) fluorene. Such a compound may be an ester-forming derivative, and is preferably a C₁₋₄alkyl ester such as a methyl ester and an ethyl ester and most preferably a C₁₋₄alkyl ester such as a methyl ester.

These fluorene-containing dicarboxylic acid components may be used alone or in combination of two or more. Among the dicarboxylic acid components represented by the formula (1), preferred is the following: a 9, 9-bis(carboxy-C₂₋₄alkyl) fluorene such as 9, 9-bis(2-carboxyethyl) fluorene and 9, 9-bis(2-carboxypropyl) fluorene; a 9, 9-bis(carboxy-C₂₋₄alkyl)-2, 7-diphenylfluorene such as 9, 9-bis(2-carboxyethyl)-2, 7-diphenylfluorene and 9, 9-bis(2-carboxypropyl)-2, 7-diphenylfluorene; or a 9, 9-bis(carboxy-C₂₋₄alkyl)-2, 7-dinaphthylfluorene such as 9, 9-bis(2-carboxyethyl)-2,7-dinaphthylfluorene and 9, 9-bis(2-carboxypropyl)-2,7-dinaphthylfluorene. From the viewpoint of easy recovery of the monomer component by depolymerization of the fluorene-containing resin, the 9, 9-bis(carboxy-C₂₋₄alkyl)-2,7-dinaphthylfluorene is particularly preferred. In the 9, 9-bis(carboxy-C₂-4alkyl)-2,7-dinaphthylfluorene, each naphthyl group may be 1-naphthyl group (the component may be a 9,9-bis(carboxy-C₂₋₄alkyl)-2,7-di(1-naphthyl) fluorene), and is preferably 2-naphthyl group (the component is preferably a 9, 9-bis(carboxy-C₂₋₄alkyl)-2,7-di(2-naphthyl) fluorene such as 9, 9-bis(2-carboxyethyl)-2,7-di(2-naphthyl) fluorene). Such a compound may be an ester-forming derivative, and is preferably a C₁-alkyl ester such as a methyl ester and an ethyl ester and most preferably a C₁₋₄alkyl ester such as a methyl ester.

The fluorene-containing dicarboxylic acid component represented by the formula (1) and the process for producing the component are known. The compound of the formula (1) in which each of n1 and n2 is 0 can be prepared by allowing a 9H-fluorene to react with components corresponding to the groups [—A¹—CO—X¹] and [—A²—CO—X²], for example, (meth)acrylic acid or an ester thereof, in accordance with the method described in Japanese Patent Application Laid-Open Publication No. 2005-89422 (JP 2005-89422 A). The compound of the formula (1) in which each of n1 and n2 is not less than 1 can be prepared by subjecting a fluorene skeleton-containing compound having the groups [—A¹—CO—X′] and [—A²—CO—X²] at 9, 9-positions to coupling reaction with a compound having an arene ring corresponding to the ring Z¹ and a compound having an arene ring corresponding to the ring Z², in accordance with the method described in WO 2020/213470; or by coupling a compound having an arene ring corresponding to the ring Z and a compound having an arene ring corresponding to the ring Ze with a benzene ring of a 9H-fluorene, and allowing the resulting compound (the 9H-fluorene skeleton-containing compound) to react with components corresponding to the groups [—A¹—CO—X¹] and [—A²—CO—X²], for example, (meth)acrylic acid or an ester thereof, using the method described in JP 2005-89422 A.

### (Fluorene-Containing Diol)

In a case where the diol component as a polymerization component contains a fluorene skeleton-containing diol (a diol with a fluorene skeleton or a fluorene-containing diol), the fluorene-containing diol is not particularly limited to a specific one, and is preferably a diol represented by the formula (2),

In the formula (2), the arene rings represented by Z³ and Z⁴ may include, for example, the same arene rings as the rings Z¹ and Z² of the formula (1). The species of the rings Z³ and Z⁴ may be the same or different from each other and are usually the same. Among the rings Z³ and Z⁴, preferred is a C₆₋₁₂arene ring such as a benzene ring, a naphthalene ring, and a biphenyl ring, and further preferred is a C₆₋₁₀arene ring such as a benzene ring and a naphthalene ring.

The bonding positions of the rings Ze and Z⁴ with respect to 9-position of the fluorene ring are not particularly limited to specific positions. For example, in a case where each of the rings Z³ and Z⁴ is a naphthalene ring, the bonding position is 1-position or 2-position, preferably 2-position; in a case where each of the rings Z³ and Z⁴ is a biphenyl ring, the bonding position is any position of 2-position, 3-position, and 4-position, and preferably 3-position.

The substituent R⁷ may be a non-reactive substituent inert or inactive to a reaction, and may include, for example, a cyano group; a halogen atom such as a fluorine atom, a chlorine atom, and a bromine atom; and a hydrocarbon group such as an alkyl group and an aryl group. Examples of the aryl group may include a C₆₋₁₀aryl group such as phenyl group. The preferred group R⁷ is the cyano group, the halogen atom, or the alkyl group, and particularly the alkyl group. The alkyl group may include, for example, a C₁₋₁₂alkyl group such as methyl group, ethyl group, n-propyl group, isopropyl group, n-butyl group, and t-butyl group, preferably a C₁₋₈alkyl group, further preferably a C₁₋₈alkyl group, and more preferably a C₁₋₄alkyl group such as methyl group.

In a case where the substitution number u of the groups R⁷ is 2 or more, the species of the two or more groups R⁷ bonded to one or the other of the two benzene rings constituting the fluorene ring may be the same or different from each other; and the species of the two or more groups R⁷ bonded to each of the two benzene rings may be the same or different from each other. The bonding position(s) (substitution position(s)) of the group(s) R⁷ is any of 1- to 8-positions of the fluorene ring without particular limitation, and may for example be 2-position, 7-position, and 2, 7-positions of the fluorene ring.

The substitution number u may for example be an integer of 0 to 6. The substitution number u is preferably an integer of 0 to 4, an integer of 0 to 3, an integer of 0 to 2, and 0 or 1 in a stepwise manner, and most preferably 0. The number of the substituents R⁷ on one of the two benzene rings constituting the fluorene ring may be different from the number of the substituents R⁷ on the other, and the same substitution numbers are preferred.

The substituents represented by R⁵ and R⁶ may include, for example, the same as the substituents exemplified as R¹ and R² in the formula (1) (such as the halogen atom; the alkyl group, the aryl group; the alkoxy group; the acyl group; the nitro group; the cyano group; and the mono- or di-substituted amino group), a cycloalkyl group, and an aralkyl group.

The preferred groups R⁵ and R⁶ are each the alkyl group, the aryl group, and the alkoxy group, and further preferably include a C₁₋₆alkyl group such as methyl group, a C₆₋₁₄aryl group such as phenyl group, and a C₁₋₄alkoxy group such as methoxy group. Among these substituents, the alkyl group or the aryl group is preferred, and a C₁₋₄alkyl group such as methyl group or a C₆₋₁₀aryl group such as phenyl group is particularly preferred. In a case where each of the group R¹ or R² is the aryl group, the group R⁵ or R⁶ may form the ring-assemblies arene ring together with the ring Z³ or Z⁴.

Each of the substitution numbers t1 and t2 is an integer of not less than 0, and may be selected according to the species of the ring Z³ or Z⁴. The substitution number may for example be an integer of 0 to 8. The substitution number is preferably an integer of 0 to 4, an integer of 0 to 3, an integer of 0 to 2, and 0 or 1 in a stepwise manner, and most preferably 0. In a case where each of the substitution numbers t1 and t2 is an integer of not less than 2, the species of two or more groups R⁵ and R⁶ may be the same or different from each other.

In a case where each of the substitution numbers t1 and t2 is 1, each of the rings Z³ and Z⁴ may represent a benzene ring, a naphthalene ring, or a biphenyl ring, and each of the groups R⁵ and R⁶ may represent methyl group. In a case where each of t1 and t2 denotes 2, each of the rings Z² and Zª may represent a benzene ring, and each of the groups R⁵ and R⁶ may represent methyl group. The substitution positions of the groups R⁵ and R⁶ are not particularly limited to specific positions. The group R⁵ or R⁶ is usually substituted at least ortho-position of the ether bond-containing group [—O—(A³O)s1—H] or [—O—(A⁴O)s2—H] (or substituted at the carbon atom adjacent to the bonding position of the ether bond-containing group) in the ring Z³ or Z⁴.

The alkylene groups A³ and A⁴ may include, for example, a C₂₋₆alkylene group such as ethylene group, propylene group (1, 2-propanediyl group), trimethylene group, 1, 2-butanediyl group, and tetramethylene group, and are preferably a C₂₋₄alkylene group, further preferably a C₂₋₃alkylene group such as ethylene group and propylene group, and most preferably ethylene group.

Each of the repeating numbers s1 and s2 is not less than 0, and may for example be selected from a range of an integer of 0 to 15. In order to accelerate an esterification reaction, the repeating number is not less than 1, and is preferably an integer of 1 to 10, an integer of 1 to 8, an integer of 1 to 6, an integer of 1 to 4, an integer of 1 to 3, and 1 or 2 in a stepwise manner, and most preferably 1. The repeating numbers s1 and s2 may be the same or different from each other; in a case where each of s1 and s2 is an integer of not less than 2, the species of the two or more alkylene groups A³ and A⁴ may be the same or different from each other. In this description and claims, the term “repeating number (added mole number)” may be an average (arithmetic average, arithmetical average) or an average addition molar amount (an average number of moles added), and preferred aspects may satisfy the above-mentioned preferred ranges (the above-mentioned integer ranges).

The substitution positions of the groups [—O—(A³O)s1-] and [—O—(A⁴O)s2-] (each of which may be referred to as an ether-containing group) on the rings Z³ and Z⁴ are not particularly limited to specific positions. In a case where each of the rings Z³ and Z⁴ is a benzene ring, each of the ether-containing groups is practically substituted at any of 2-position, 3-position, and 4-position of each phenyl group bonded to 9-position of the fluorene ring, preferably 3-position or 4-position, and particularly 4-position; in a case where each of the rings Z³ and Z⁴ is a naphthalene ring, 1-position or 2-position of each naphthalene ring is bonded to 9-position of the fluorene ring (the fluorene ring has 1-naphthyl or 2-naphthyl substituent), and each ether-containing group is practically substituted at each naphthalene ring at a relationship of 1,5-positions or 2,6-positions, particularly 2, 6-positions with respect to the bonding position. In a case where each of the rings 23 and 24 is a biphenyl ring (or each of the rings 23 and 24 is a benzene ring, each of t1 and t2 is 1, and each of R⁵ and R⁶ is phenyl group), 3-position or 4-position of the biphenyl ring may be bonded to 9-position of the fluorene ring. In a case where 3-position of the biphenyl ring is bonded to 9-position of the fluorene ring, the substitution position of the ether-containing group may be 6-position or 4′-position of the biphenyl ring, particularly 6-position.

Examples of the fluorene-containing diol may include a 9, 9-bis(hydroxyaryl) fluorene represented by the formula (2) in which each of s1 and s2 is 0; and a 9, 9-bis[hydroxy(poly)alkoxyaryl]fluorene represented by the formula (2) in which each of s1 and s2 is not less than 1, for example, 1 to 10.

The preferred fluorene-containing diol may contain a compound represented by the following formula (2a) or (2b):

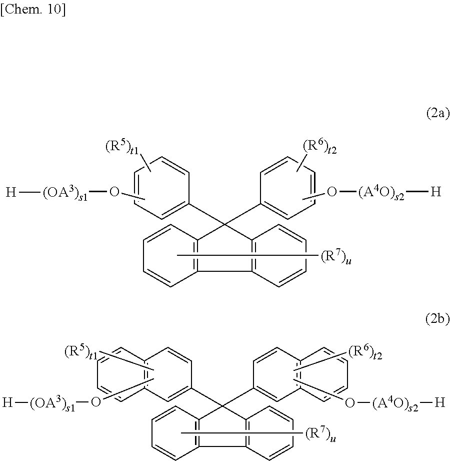

wherein A³, A⁴, s1, s2, R⁵, R⁶, t1, t2, R, and u each have the same meanings as defined in the formula (2).

Examples of a 9, 9-bis(hydroxyphenyl) fluorene corresponding to the formula (2a) may include a 9, 9-bis(hydroxyphenyl) fluorene such as 9, 9-bis(4-hydroxyphenyl) fluorene; a 9, 9-bis(alkyl-hydroxyphenyl) fluorene, specifically a 9, 9-bis[(mono- or di-) C₁₋₄alkyl-hydroxyphenyl]fluorene such as 9, 9-bis(4-hydroxy-3-methylphenyl) fluorene, 9, 9-bis(4-hydroxy-3-isopropylphenyl) fluorene, and 9, 9-bis(4-hydroxy-3, 5-dimethylphenyl) fluorene; and a 9, 9-bis(aryl-hydroxyphenyl) fluorene, specifically a 9,9-bis(C₆₋₁₀aryl-hydroxyphenyl) fluorene such as 9, 9-bis(4-hydroxy-3-phenylphenyl) fluorene.

Examples of a 9,9-bis[hydroxy(poly)alkoxyphenyl]fluorene corresponding to the formula (2a) may include an alkylene oxide (or an alkylene carbonate, a haloalkanol) adduct of the 9, 9-bis(hydroxyphenyl) fluorene, for example, a 9, 9-bis[hydroxy(poly)alkoxyphenyl]fluorene, specifically a 9, 9-bis[hydroxy (mono-to deca-) C₂₋₄alkoxy-phenyl]fluorene such as 9, 9-bis[4-(2-hydroxyethoxy)phenyl]fluorene, 9, 9-bis[4-(2-(2-hydroxyethoxy) ethoxy)phenyl]fluorene, and 9, 9-bis[4-(2-hydroxypropoxy)phenyl]fluorene; a 9, 9-bis[alkyl-hydroxy(poly)alkoxyphenyl]fluorene, specifically a 9, 9-bis[(mono- or di-) C₁₋₄alkyl-hydroxy (mono-to deca-) C₂₋₄alkoxy-phenyl]fluorene such as 9, 9-bis[4-(2-hydroxyethoxy)-3-methylphenyl]fluorene, 9, 9-bis[4-(2-(2-hydroxyethoxy) ethoxy)-3-methylphenyl]fluorene, 9, 9-bis[4-(2-hydroxyethoxy)-3, 5-dimethylphenyl]fluorene, and 9, 9-bis[4-(2-hydroxypropoxy)-3-methylphenyl]fluorene; and a 9, 9-bis[aryl-hydroxy(poly)alkoxyphenyl]fluorene, specifically a 9, 9-bis[C₆₋₁₀aryl-hydroxy (mono-to deca-) C₂₋₄alkoxy-phenyl]fluorene such as 9, 9-bis(4-(2-hydroxyethoxy)-3-phenylphenyl) fluorene, 9, 9-bis[4-(2-(2-hydroxyethoxy) ethoxy)-3-phenylphenyl]fluorene, and 9, 9-bis(4-(2-hydroxypropoxy)-3-phenylphenyl) fluorene.

Examples of a 9, 9-bis(hydroxynaphthyl) fluorene corresponding to the formula (2b) may include a 9,9-bis(hydroxynaphthyl) fluorene such as 9, 9-bis(6-hydroxy-2-naphthyl) fluorene and 9, 9-bis(5-hydroxy-1-naphthyl) fluorene; examples of a 9, 9-bis[hydroxy(poly)alkoxynaphthyl]fluorene corresponding to the formula (2b) may include a 9,9-bis[hydroxy (mono-to deca-) C₂₋₄alkoxy-naphthyl]fluorene such as 9, 9-bis[6-(2-hydroxyethoxy)-2-naphthyl]fluorene, 9, 9-bis[5-(2-hydroxyethoxy)-1-naphthyl]fluorene, 9, 9-bis[6-(2-(2-hydroxyethoxy) ethoxy)-2-naphthyl]fluorene and 9, 9-bis[6-(2-hydroxypropoxy)-2-naphthyl]fluorene.

These fluorene-containing diols may be used alone or in combination of two or more. Among them, preferred is a 9,9-bis[hydroxy(poly)alkoxyaryl]fluorene such as a 9, 9-bis[hydroxy (mono-to hexa-) C₂₋₄alkoxy-C₆₋₁₂aryl]fluorene, further preferred is a 9, 9-bis[hydroxy (mono- or di-) C₂₋₄alkoxy-C₆₋₁₂aryl]fluorene, and more preferred is a 9,9-bis[hydroxy-C₂₋₃alkoxy-C₆₋₁₂aryl]fluorene such as 9, 9-bis[4-(2-hydroxyethoxy)phenyl]fluorene (BPEF), 9, 9-bis[4-(2-hydroxyethoxy)-3-methylphenyl]fluorene, 9, 9-bis[4-(2-hydroxyethoxy)-3, 5-dimethylphenyl]fluorene, 9, 9-bis[4-(2-hydroxyethoxy)-3-phenylphenyl]fluorene (BOPPEF), and 9, 9-bis[6-(2-hydroxyethoxy)-2-naphthyl]fluorene (BNEF). Among them, BPEF, BOPPEF, or BNEF is particularly preferred, and BPEF or BNEF is most preferred.

### (Other Dicarboxylic Acid Components)

The dicarboxylic acid component may contain another dicarboxylic acid component (a dicarboxylic acid component other than the fluorene-containing dicarboxylic acid component) in addition to the fluorene-containing dicarboxylic acid component. In a case where the diol component contains the fluorene-containing diol, the dicarboxylic acid component may be another dicarboxylic acid component alone.

Examples of another dicarboxylic acid component may include an aliphatic dicarboxylic acid component, an alicyclic dicarboxylic acid component, and an aromatic dicarboxylic acid component.

Examples of the aliphatic dicarboxylic acid component may include an alkanedicarboxylic acid and an unsaturated aliphatic dicarboxylic acid, or an ester-forming derivative of such a dicarboxylic acid. Examples of the alkanedicarboxylic acid may include a C₁₋₂₀alkane-dicarboxylic acid such as malonic acid, succinic acid, and adipic acid. Examples of the unsaturated aliphatic dicarboxylic acid may include a C₂₋₁₀alkene-dicarboxylic acid such as maleic acid and fumaric acid.

Examples of the alicyclic dicarboxylic acid component may include a cycloalkanedicarboxylic acid, a bridged ring (crosslinked ring) cycloalkanedicarboxylic acid, a cycloalkenedicarboxylic acid, and a bridged ring (crosslinked ring) cycloalkenedicarboxylic acid, or an ester-forming derivative of such a dicarboxylic acid. Examples of the cycloalkanedicarboxylic acid may include a C₄₋₁₂cycloalkane-dicarboxylic acid such as cyclohexanedicarboxylic acid. Examples of the bridged ring cycloalkanedicarboxylic acid may include a (bi- or tri-)cycloC₇₋₁₀alkane-dicarboxylic acid such as norbornanedicarboxylic acid. Examples of the cycloalkenedicarboxylic acid may include a C₅₋₁₀cycloalkene-dicarboxylic acid such as cyclopentenedicarboxylic acid. Examples of the bridged ring cycloalkenedicarboxylic acid may include a (bi- or tri-) cycloC₇₋₁₀alkene-dicarboxylic acid such as norbornenedicarboxylic acid.

Examples of the aromatic dicarboxylic acid component may include a monocyclic aromatic dicarboxylic acid and a polycyclic aromatic dicarboxylic acid, or an ester-forming derivative of such a dicarboxylic acid. Examples of the monocyclic aromatic dicarboxylic acid component may include a benzenedicarboxylic acid such as phthalic acid, isophthalic acid, and terephthalic acid; and a C₁₋₄alkyl-benzenedicarboxylic acid such as 5-methylisophthalic acid. Examples of the polycyclic arenedicarboxylic acid component may include a condensed polycyclic arenedicarboxylic acid and a ring-assemblies arenedicarboxylic acid. Examples of the condensed polycyclic arenedicarboxylic acid may include a condensed polycyclic C₁₀₋₂₄arene-dicarboxylic acid, e.g., a naphthalenedicarboxylic acid such as 1, 2-naphthalenedicarboxylic acid, 1, 5-naphthalenedicarboxylic acid, 1, 8-naphthalenedicarboxylic acid, and 2, 6-naphthalenedicarboxylic acid; an anthracenedicarboxylic acid; and a phenanthrenedicarboxylic acid. Examples of the ring-assemblies arenedicarboxylic acid may include may include a biC₆₋₁₀arene-dicarboxylic acid such as 2, 2′-biphenyldicarboxylic acid, 3,3′-biphenyldicarboxylic acid, and 4, 4′-biphenyldicarboxylic acid.

These other dicarboxylic acid components may be used alone or in combination of two or more. Among them, the aromatic dicarboxylic acid component is preferred, the benzenedicarboxylic acid such as terephthalic acid is particularly preferred.

### (Other Diol Components)

The diol component may contain another diol component (a diol component other than the fluorene-containing diol) in addition to the fluorene-containing diol. In a case where the dicarboxylic acid component contains the fluorene-containing dicarboxylic acid component, the diol component may be another diol component alone,

Examples of another diol component may include a chain aliphatic diol, an alicyclic diol, and an aromatic diol.

Examples of the chain aliphatic diol may include a C₂₋₁₀alkanediol such as ethylene glycol, 1, 2-propanediol, 1,3-propanediol, 1, 2-butanediol, 1, 3-butanediol, 1,4-butanediol, 1,3-pentanediol, 1, 4-pentanediol, 1,5-pentanediol, and neopentyl glycol; and a di- or tri-C₂₋₄alkanediol such as diethylene glycol, dipropylene glycol, and triethylene glycol.

Examples of the alicyclic diol may include a C₅₋₈cycloalkanediol such as cyclohexanediol; and a di(hydroxy-C₁₋₄alkyl) C₅₋₈cycloalkane such as cyclohexanedimethanol.

Examples of the aromatic diol may include a dihydroxyarene such as hydroquinone and resorcinol; an araliphatic diol such as benzenedimethanol; a bisphenol such as bisphenol F, bisphenol AD, bisphenol A, bisphenol C, bisphenol G, and bisphenol S; a biphenol such as p, p′-biphenol; a binaphthol such as binaphthol; and a C₂₋₄alkylene oxide (or an alkylene carbonate, a haloalkanol) adduct of such a diol component. The binaphthol may be a diol represented by the after-mentioned formula (5).

These other diol components may be used alone or in combination of two or more. Among them, a low molecule weight aliphatic diol (a chain aliphatic diol) such as the alkanediol is preferred, and a C₂₋₄alkanediol such as ethylene glycol is further preferred.

### (Carbonate Ester Bond-Forming Component)

In a case where the fluorene-containing polyester-series resin is a fluorene-containing polyester-carbonate ester resin, the resin contains a carbonate ester bond-forming component as a polymerization component for forming a carbonate ester unit, in addition to the dicarboxylic acid component and the diol component. In the description and the claims of this application, the term “carbonate ester unit” means a constitutional unit derived from a carbonate ester bond-forming component, that is, means to contain a carbonyl group [—C(═O)—], and the carbonyl group forms a carbonate ester bond together with terminal oxygen atoms of constitutional units derived from two diol components adjacently bonded to the carbonyl group. Thus, any compound that can form a carbonate ester bond by a reaction with two diol components may be used as the carbonate ester bond-forming component. The representative carbonate ester bond-forming component may include, for example, a phosgene such as phosgene and triphosgene, and a carbonate diester such as a diphenylcarbonate ester. From the viewpoint of safety and others, the carbonate diester such as the diphenylcarbonate ester is preferred.

In the fluorene-containing polyester-carbonate ester resin, the ratio of the total amount of the dicarboxylic acid component and the carbonate ester bond-forming component relative to the diol component is 1/0.8 to 1/1.2 and preferably 1/0.9 to 1/1.1 in terms of the former/the latter (molar ratio), and is preferably substantially equimolar. The ratio of the dicarboxylic acid component relative to the carbonate ester bond-forming component may be selected from a range of 99/1 to 1/99 in terms of the former/the latter (molar ratio), and is preferably 95/5 to 10/90, 90/10 to 20/80, 80/20 to 30/70, and 70/30 to 40/60 in a stepwise manner. In a case where the ratio of the carbonate ester bond-forming component is excessively high, the refractive index or the heat resistance may be lowered.

### (Characteristics of Fluorene-Containing Polyester-Series Resin)

In the fluorene-containing polyester-series resin, the proportion of the fluorene skeleton-containing polymerization component in the whole polymerization component containing the dicarboxylic acid component and the diol component may be not less than 10% by mol, and is preferably not less than 30% by mol, 30 to 99% by mol, 40 to 98% by mol, 50 to 95% by mol, 70 to 93% by mol, 80 to 90% by mol, and 82 to 88% by mol in a stepwise manner. The present disclosure allows efficient depolymerization of even a fluorene-containing polyester-series resin with a fluorene skeleton in a high ratio, in particular, a fluorene-containing polyester resin, and allows efficient recovery of a useful fluorene skeleton-containing monomer component (or monomer).

In the description and the claims of this application, the ratio (molar ratio) of a polymerization component means the ratio (molar ratio) as a constitutional unit in the fluorene-containing resin.

The weight-average molecular weight of the fluorene-containing polyester-series resin may for example be selected from a range of about 15,000 to 100,000, and is, for example, 20,000 to 80,000, preferably 30,000 to 70,000, further preferably 40,000 to 65,000, and most preferably 45,000 to 60,000.

In the description and the claims of this application, the weight-average molecular weight of the fluorene-containing polyester-series resin can be measured by gel permeation chromatography using a polystyrene as a standard material.

The glass transition temperature (Tg) of the fluorene-containing polyester-series resin may for example be selected from a range of about 100 to 250° C., and is, for example, 110 to 230° C., preferably 120 to 210° C., further preferably 130 to 200° C., and most preferably 135 to 190° C. In the description and the claims of this application, the glass transition temperature can be measured using a differential scanning calorimeter, specifically can be measured according to the method described in the after-mentioned Examples.

The fluorene-containing polyester-series resin may be amorphous.

In the fluorene-containing polyester-series resin such as the fluorene-containing polyester resin, the proportion of the fluorene-containing dicarboxylic acid component is not particularly limited to a specific one. In a case where the diol component contains the fluorene-containing diol, the dicarboxylic acid component does not necessarily contain the fluorene-containing dicarboxylic acid component. The proportion of the fluorene-containing dicarboxylic acid component in the dicarboxylic acid component is preferably not less than 10% by mol, and is preferably not less than 30% by mol, not less than 50% by mol, not less than 708 by mol, not less than 80% by mol, and not less than 90% by mol in a stepwise manner. The most preferred proportion is 100% by mol.

The proportion of the fluorene-containing diol is not particularly limited to a specific one, In a case where the dicarboxylic acid component contains the fluorene-containing dicarboxylic acid component, the diol component does not necessarily contain the fluorene-containing diol. The proportion of the fluorene-containing diol in the diol component is preferably not less than 1% by mol, and is preferably not less than 5% by mol, 10 to 99% by mol, 30 to 95% by mol, 40 to 90% by mol, and 50 to 85% by mol in a stepwise manner. The most preferred proportion is 60 to 80% by mol.

In the fluorene-containing polyester-series resin such as the fluorene-containing polyester resin, the dicarboxylic acid component and/or the diol component has a fluorene skeleton. It is preferred that at least the diol component contain a fluorene skeleton, and it is most preferred that both dicarboxylic acid component and diol component contain a fluorene skeleton. A polyester-series resin in which both dicarboxylic acid component and diol component have a fluorene skeleton has a high density of the bulky fluorene skeleton, and is quite different in not only structure but also behavior from a widely used polyester-series resin. The depolymerization method of the present disclosure allows efficient recovery of such a useful fluorene skeleton-containing monomer.

In particular, the diol component preferably contains not only the fluorene-containing diol but also the fluorene-containing diol in combination with another diol component (particularly an aliphatic diol such as a C₂₋₄alkanediol).

In the fluorene-containing polyester-series resin such as the fluorene-containing polyester resin, the molar ratio of the fluorene-containing diol relative to another diol component (in particular, the aliphatic diol) may be selected from a range of about 100/0 to 30/70 in terms of the former/the latter, and a preferred range of the molar ratio is 99/1 to 40/60, 95/5 to 50/50, 90/10 to 55/45, and 80/20 to 60/40 in a stepwise manner. The most preferred molar ratio is 75/25 to 65/35 in terms of the former/the latter.

### (A) First Decomposition Step

The depolymerization method for the fluorene-containing polyester-series resin of the present disclosure includes a first decomposition step of allowing the fluorene-containing polyester-series resin to react with a carbonate ester in the presence of a first hydrolysis catalyst to decompose the fluorene-containing polyester-series resin, obtaining a decomposition product (a first decomposition product) containing a dicarboxylic acid and/or an ester thereof, and a monocarbonate ester form of a diol and/or a dicarbonate ester form of a diol,

### (Carbonate Ester)

The carbonate ester serves as a depolymerization agent of the fluorene-containing polyester-series resin. Examples of the carbonate ester may include a carbonate diester such as a dialkyl carbonate (a dialkanol carbonate ester) and a diaryl carbonate, Examples of the dialkanol carbonate ester may include dimethyl carbonate, diethyl carbonate, dipropyl carbonate, diisopropyl carbonate, and di-t-butyl carbonate. Example of the diaryl carbonate may include diphenyl carbonate and toluylphenyl carbonate,

These carbonate esters may be used alone or in combination of two or more. Among them, a diC₁₋₄alkyl carbonate is preferred, a diC₁₋₃alkyl carbonate is further preferred, a diC₁₋₂alkyl carbonate is more preferred, and dimethyl carbonate is most preferred.

The ratio of the carbonate ester relative to 1 mol of the fluorene-containing polyester-series resin (the number of moles corresponding to number-average molecular weight) may be not less than 1 mol, and is, for example, 1 to 50 mol, preferably 2 to 30 mol, further preferably 3 to 20 mol, more preferably 5 to 15 mol, and most preferably 8 to 12 mol. The ratio of the carbonate ester relative to 100 parts by mass of the fluorene-containing polyester-series resin may for example be not less than 50 parts by mass, and is, for example, 50 to 1000 parts by mass, preferably 100 to 500 parts by mass, further preferably 120 to 400 parts by mass, more preferably 150 to 380 parts by mass, and most preferably 180 to 350 parts by mass. In a case where the ratio of the carbonate ester is excessively low, the depolymerization may not proceed rapidly.

### (First Hydrolysis Catalyst)

The first hydrolysis catalyst is a conventional hydrolysis catalyst, and may be an acid catalyst and is preferably an alkali catalyst. The alkali catalyst may include, for example, an inorganic base and an organic base.

Examples of the inorganic base may include an alkali metal hydroxide such as lithium hydroxide, sodium hydroxide, and potassium hydroxide; an alkaline earth metal hydroxide such as magnesium hydroxide, calcium hydroxide, and barium hydroxide; an alkali metal oxide such as sodium oxide and potassium oxide; an alkaline earth metal oxide such as magnesium oxide, calcium oxide, and barium oxide; an alkali or alkaline earth metal carbonate such as cesium carbonate and sodium carbonate; an alkali or alkaline earth metal hydrogen carbonate such as sodium hydrogen carbonate; and ammonia.

Examples of the organic base may include an alkali or alkaline earth metal carboxylate such as sodium acetate and calcium acetate; an alkali metal alkoxide such as lithium methoxide, sodium methoxide, potassium methoxide, sodium ethoxide, sodium propoxide, and sodium t-butoxide; an organic metal compound such as butyllithium, phenyllithium, isopropylmagnesium chloride, cyclohexylmagnesium bromide, phenylmagnesium bromide, sodium amide, and lithium diisopropylamide; and an amine.

These alkali catalysts may be used alone or in combination of two or more. Among them, an alkali metal C₁₋₆alkoxide is preferred, an alkali metal C₁₋₄alkoxide is further preferred, an alkali metal C₁₋₃alkoxide is more preferred, and an alkali metal C₁₋₂alkoxide such as a lithium C₁₋₂alkoxide is most preferred.

The ratio of the first hydrolysis catalyst relative to 1 mol of the fluorene-containing polyester-series resin may for example be not less than 0.01 mol, and is preferably 0.01 to 0.3 mol, further preferably 0.03 to 0.2 mol, more preferably 0.05 to 0.15 mol, and most preferably 0.08 to 0.12 mol. The ratio of the first hydrolysis catalyst relative to 100 parts by mass of the fluorene-containing polyester-series resin may for example be not less than 0.1 parts by mass, and is preferably 0.1 to 5 parts by mass, further preferably 0.2 to 3 parts by mass, more preferably 0.3 to 2 parts by mass, and most preferably 0.5 to 1.5 parts by mass. In a case where the ratio of the first hydrolysis catalyst is excessively low, the depolymerization may not proceed rapidly. The ratio of the first hydrolysis catalyst may be the ratio of the alkali catalyst.

### (Reaction Condition)

The reaction temperature may be not lower than 20° C., and is, for example, 20 to 85° C., preferably 25 to 83° C., further preferably 30 to 80° C., more preferably 50 to 75° C., and most preferably 60 to 70° C.

The reaction time may be not less than 10 minutes, and is, for example, 10 minutes to 10 hours, preferably 30 minutes to 8 hours, further preferably 1 to 5 hours, and more preferably 2 to 4 hours,

The reaction may be carried out in the presence of an inert or inactive gas. From the viewpoint of convenience and others, the reaction is preferably carried out in the atmosphere. Moreover, the reaction may be carried out under a reduced pressure. From the viewpoint of convenience and others, the reaction is preferably carried out under the atmospheric pressure.

### (First Decomposition Product)

The first decomposition product in the first decomposition step contains a dicarboxylic acid and/or an ester thereof, and a monocarbonate ester form of a diol and/or a dicarbonate ester form of a diol. Thus, the present disclosure also includes a process for producing a monocarbonate ester form of a diol and/or a dicarbonate ester form of a diol by the first decomposition step.

The dicarboxylic acid ester may be an alkyl ester form, As the alkyl ester form, a C₁₋₃alkyl ester form such as a methyl ester form and an ethyl ester form is preferred, and the methyl ester form is particularly preferred. In a case where the dicarboxylic acid and/or the ester thereof is a dicarboxylic acid component represented by the formula (1), the dicarboxylic acid and/or the ester thereof may be a dicarboxylic acid component of the formula (1) in which each of X¹ and X² represents a C₁₋₄alkoxy group, and is preferably a dicarboxylic acid component represented by the formula (1) in which each of X¹ and X² represents a C₁₋₂alkoxy group such as methoxy group, particularly preferably each of X¹ and X² represents methoxy group. In a case where each of X¹ and X⁴ represents a C₁₋₂alkoxy group, a newly produced fluorene-containing resin can be obtained at a high productivity in chemical recycling. Each of the alkyl groups contained in the alkoxy groups X¹ and X² may be an alkyl group derived from the alkali catalyst.

The monocarbonate ester form of the diol and/or the dicarbonate ester form of the diol may be converted into a diol by subjecting to the next second decomposition step, and the resulting diol may be reused as a raw material for a fluorene-containing polyester-series resin. The monocarbonate ester form of the diol and/or the dicarbonate ester form of the diol may be used as a carbonate ester compound without being subjected to the second decomposition step.

Examples of the monocarbonate ester form of the diol may include a monocarbonate ester form of a diol represented by the formula (3). This monocarbonate ester form is a novel compound and can be used as a raw material for a polycarbonate ester-series resin, a reaction conditioner, a resin additive, and others.

The monocarbonate ester form of the diol represented by the formula (3) is a monocarbonate ester form in which a carbonate ester is added to either one hydroxyl group of the diol represented by the formula (2).

Examples of the hydrocarbon group represented by R⁸ in the formula (3) may include an alkyl group and an aryl group. Among them, the alkyl group or phenyl group is preferred, and the alkyl group is particularly preferred. Examples of the alkyl group may include methyl group, ethyl group, propyl group, isopropyl group, and butyl group. These alkyl groups may be used alone or in combination of two or more. Among them, a C₁₋₄alkyl group is preferred, a C₁₋₃alkyl group is further preferred, a C₁₋₂alkyl group is more preferred, and methyl group is most preferred.

Examples of the dicarbonate ester form of the diol may include a dicarbonate ester form of a diol represented by the formula (4). This dicarbonate ester form is also a novel compound and can be used as a raw material for a polycarbonate ester-series resin, a resin additive, and others.

The dicarbonate ester form of the diol represented by the formula (4) is a dicarbonate ester form in which carbonate esters are added to both hydroxyl groups of the diol represented by the formula (2).

Examples of the hydrocarbon groups represented by R⁹ and R¹⁰ in the formula (4) may include an alkyl group and an aryl group. Among them, the alkyl group or phenyl group is preferred, and the alkyl group is particularly preferred. Examples of the alkyl group may include methyl group, ethyl group, propyl group, isopropyl group, and butyl group. These alkyl groups may be used alone or in combination of two or more. Among them, a C₁₋₄alkyl group is preferred, a C₁₋₃alkyl group is further preferred, a C₁₋₂alkyl group is more preferred, and methyl group is most preferred.

In the first decomposition step, the total ratio of the monocarbonate ester form and the dicarbonate ester form relative to 1 mol of the dicarboxylic acid component is, for example, 0.1 to 5 mol, preferably 0.2 to 3 mol, further preferably 0.3 to 2 mol, and more preferably 0.5 to 1.5 mol.

The ratio of the monocarbonate ester form relative to 1 mol of the dicarbonate ester form is, for example, 0.1 to 1 mol, preferably 0.2 to 0.9 mol, further preferably 0.3 to 0.8 mol, and more preferably 0.4 to 0.6 mol.

The first decomposition product may further contain a diol. The diol may be a diol corresponding to the monocarbonate ester form or the dicarbonate ester form. The ratio of the diol relative to 1 mol of the total amount of the dicarboxylic acid and the ester thereof is about not more than 0.2 mol, and is, for example, not more than 0.1 mol, preferably not more than 0.05 mol, and further preferably 0.01 to 0.03 mol.

The first decomposition product may undergo a conventional purification treatment. The conventional purification treatment may include, for example, optionally subjecting a mixture containing the decomposition product to neutralization and washing with water, and then removing impurities from the mixture by filtration or other means. In a case where the monomer component in the mixture is precipitated, the monomer component may completely be dissolved using a good solvent for the monomer component and then the resulting mixture may be subjected to neutralization and washing with water.

In the first decomposition step, the dicarboxylic acid and/or the ester thereof, and the monocarbonate ester form of the diol and/or the dicarbonate ester form of the diol are mainly obtained. Thus, in a case where the objective fluorene skeleton-containing monomer component is a dicarboxylic acid component or in a case where an objective compound is the novel carbonate ester compound, the depolymerization method of the present disclosure may be a depolymerization method of which decomposition step only contains the first decomposition step and does not contain the after-mentioned second decomposition step.

### (B) Second Decomposition Step

The depolymerization method for the fluorene-containing polyester-series resin of the present disclosure may have a second decomposition step for obtaining a second decomposition product, in addition to the first decomposition step. In the second decomposition step, the first decomposition product is allowed to react with an alcohol to obtain a diol. Specifically, in the second decomposition step, the monocarbonate ester form of the diol and/or the dicarbonate ester form of the diol is allowed to react with the alcohol to obtain a diol. Thus, since the second decomposition step allows the monocarbonate ester form of the diol and/or the dicarbonate ester form of the diol to be converted into a diol, the second decomposition step is particularly effective in recovering a fluorene skeleton-containing diol as a monomer component, (Alcohol)

The alcohol may include, for example, a C₁₋₄ alkanol such as methanol, ethanol, isopropanol, propanol, and butanol. These alcohols may be used alone or in combination of two or more. Among them, a C₁₋₃alkanol is preferred, a C₁₋₂alkanol is more preferred, and methanol is most preferred. Also in the second decomposition step, the decomposition reaction proceeds in presence of the dicarboxylic acid component, and the second decomposition step preferably uses an alcohol corresponding to the alkoxide used as the first hydrolysis catalyst in the first decomposition step.

The ratio of the alcohol relative to 100 parts by mass of the first decomposition product may be not less than 50 parts by mass, and is, for example, 50 to 1000 parts by mass, preferably 80 to 500 parts by mass, further preferably 100 to 300 parts by mass, more preferably 120 to 250 parts by mass, and most preferably 150 to 200 parts by mass. In a case where the ratio of the alcohol is excessively low, the yield of the diol component may be reduced.

### (Second Hydrolysis Catalyst)

Examples of a second hydrolysis catalyst may include the hydrolysis catalyst exemplified as the first hydrolysis catalyst. The second hydrolysis catalyst may be used alone or in combination of two or more. Among the second hydrolysis catalysts, the alkali metal hydroxide or the alkaline earth metal hydroxide is preferred, and the alkali metal hydroxide such as potassium hydroxide is particularly preferred.

The ratio of the second hydrolysis catalyst relative to 100 parts by mass of the first decomposition product may be not less than 1 part by mass, and is, for example, 1 to 30 parts by mass, preferably 5 to 25 parts by mass, and further preferably 10 to 20 parts by mass. In a case where the ratio of the second hydrolysis catalyst is excessively low, the yield of the diol component may be reduced. The ratio of the second hydrolysis catalyst may be the ratio of the alkali catalyst.

### (Reaction Condition)

The reaction temperature may be not lower than 20° C., and is, for example, 20 to 85° C., preferably 25 to 83° C., further preferably 30 to 80° C., more preferably 50 to 75° C., and most preferably 60 to 70° C.

The reaction time may be not less than 5 minutes, and is, for example, 5 minutes to 5 hours, preferably 10 minutes to 3 hours, further preferably 30 minutes to 2 hours, and more preferably 40 minutes to 1.5 hours.

The reaction may be carried out in the presence of an inert or inactive gas. From the viewpoint of convenience and others, the reaction is preferably carried out in the atmosphere. Moreover, the reaction may be carried out under a reduced pressure. From the viewpoint of convenience and others, the reaction is preferably carried out under the atmospheric pressure.

### (Second Decomposition Product)

The second decomposition product in the second decomposition step contains a dicarboxylic acid and/or an ester thereof, and a diol. Specifically, in a case where the depolymerization method of the present disclosure includes the second decomposition step, all raw materials of the fluorene-containing polyester-series resin can be recovered. Thus, the resulting monomer components can be used as they are to newly synthesize a fluorene-containing polyester-series resin, improving the recyclability of the fluorene-containing polyester-series resin.

### (C) Purification Step

The mixture obtained in the second decomposition step, which contains the dicarboxylic acid and/or the ester thereof, and the diol, may be separated into the dicarboxylic acid and/or the ester, and the diol by a conventional method, and each component may be subjected to a purification step.

For example, the dicarboxylic acid and/or the ester, and the diol may be separated by filtration or others using a solvent that acts as a good solvent for the diol and acts as a poor solvent for the dicarboxylic acid and/or the ester thereof. As the solvent having such a function, there may be used a C₁₋₄alkanol such as methanol.

Moreover, the dicarboxylic acid and/or the ester, and the diol may be subjected to solvent extraction using a good solvent for the dicarboxylic acid and/or the ester thereof and a solvent that is incompatible with the good solvent and that is a good solvent for the diol. The solvent extraction is effective in dissolving the dicarboxylic acid and/or the ester thereof in the good solvent for the diol, such as the C₁₋₄alkanol. Examples of the good solvent for the dicarboxylic acid and/or the ester thereof may include an aliphatic hydrocarbon such as heptane. The separated monomer components (the dicarboxylic acid and/or the ester thereof, the diol) may be purified by a conventional method. As a purification method, the conventional method such as filtration, concentration, crystallization, and column chromatography can suitably be combined. Among them, the purification method containing crystallization is preferred.

As the crystallization method, it is preferred to crystallize the monomer component with a crystallization solvent containing an aromatic hydrocarbon and/or a polar solvent.

As the aromatic hydrocarbon, preferred is a mono- or di-C₁₋₂alkyl-benzene such as toluene, xylene, and ethylbenzene, and particularly preferred is toluene.

As the polar solvent, preferred is water, a C₁₋₄alkanol such as ethanol and isopropanol, or an aliphatic ketone having 3 or more carbon atoms such as acetone and methyl isobutyl ketone, and particularly preferred is water, a C₂₋₃alkanol, or an aliphatic ketone having 4 to 8 carbon atoms.

Among the crystallization solvents, examples of the good solvent may include an aromatic hydrocarbon, and an aliphatic ketone having 4 or more carbon atoms. Examples of the poor solvent may include water and a C₁₋₈alkanol.

The ratio of the poor solvent relative to 100 parts by mass of the good solvent may for example be not more than 100 parts by mass, and is, for example, 0 to 100 parts by mass, preferably 5 to 80 parts by mass, further preferably 10 to 70 parts by mass, and more preferably 20 to 50 parts by mass.

The ratio of the crystallization solvent relative to 100 parts by mass of the monomer component is, for example, 10 to 3000 parts by mass, preferably 50 to 2000 parts by mass, further preferably 100 to 1000 parts by mass, and most preferably 200 to 500 parts by mass.

In the crystallization treatment, dissolution of the monomer component in the crystallization solvent and cooling of the resulting solution allows precipitation or crystallization of the monomer component having a higher purity. The temperature for dissolving the monomer component in the crystallization solvent is a temperature lower than the boiling point of the solvent and is, for example, 30 to 200° C., preferably 50 to 150° C., and further preferably 60 to 100° C. By a conventional method, the crystallization-treated monomer component may be washed and then dried.

[Fluorene-Containing Polycarbonate Ester-Series Resin]

The fluorene-containing polycarbonate ester-series resin of the present disclosure contains a diol component as a polymerization component, and the diol component contains a fluorene-containing diol.

### (Fluorene-Containing Diol)

The fluorene-containing diol is not particularly limited to a specific one, and is preferably the diol represented by the formula (2) described in the item of the fluorene-containing polyester-series resin. Examples of the diol represented by the formula (2) may include the diol represented by the formula (2) exemplified in the item of the fluorene-containing polyester-series resin. The diol component may be used alone or in combination of two or more,

Among the diol components, preferred is the following: a 9, 9-bis(hydroxyphenyl) fluorene such as 9, 9-bis(4-hydroxyphenyl) fluorene (BPF), 9, 9-bis[(mono- or di-) C₁₋₄alkyl-hydroxyphenyl]fluorene, and 9, 9-bis(C₆₋₁₀aryl-hydroxyphenyl) fluorene; a 9, 9-bis(hydroxynaphthyl) fluorene such as 9, 9-bis(6-hydroxy-2-naphthyl) fluorene (BNF); or a 9, 9-bis[hydroxy(poly)alkoxyaryl]fluorene such as 9, 9-bis[hydroxy (mono-to hexa-) C₂₋₄alkoxy-C₆₋₁₂aryl]fluorene, further preferred is a 9,9-bis[hydroxy (mono- or di-) C₂₋₄alkoxy-C₆₋₁₂aryl]fluorene, more preferred is a 9, 9-bis[hydroxy-C₂₋₃alkoxy-C₆₋₁₂aryl]fluorene such as BPF, BNF, BREF, 9, 9-bis[4-(2-hydroxyethoxy)-3-methylphenyl]fluorene, 9, 9-bis[4-(2-hydroxyethoxy)-3, 5-dimethylphenyl]fluorene, BOPPEF, and BNEF. Among them, BPF, BNF, BPEF, BOPPEF, or BNEF is particularly preferred, and BPEF is most preferred. (Binaphthalene-containing diol)

The diol component may contain, in addition to the fluorene-containing diol, a diol represented by the following formula (5) as a diol having a bi-(or bis-) naphthalene skeleton (a binaphthalene-containing diol):

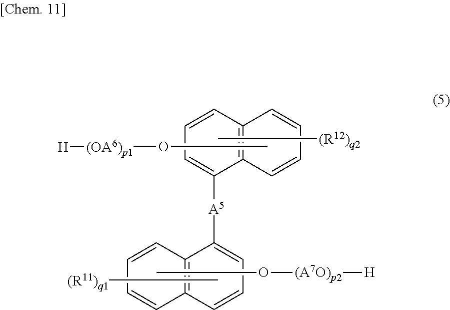

wherein
A⁵ represents a direct bond (a single bond) or an alkylene group,
A⁶ and A⁷ independently represent an alkylene group, p1 and p2 independently denote an integer of not less than 0, and
R¹¹ and R¹² independently represent a substituent, q1 and q2 independently denote an integer of 0 to 6.

In the formula (5), the substituents R¹¹ and R¹² may be selected from the same as R⁵ and R⁶ exemplified in the formula (2), including preferred aspects.

Each of the substitution number q1 of R¹¹ and the substitution number q2 of R¹² may be an integer of about 0 to 5, and is preferably an integer of 0 to 4, an integer of 0 to 3, and an integer of 0 to 2 in a stepwise manner. Each of the substitution numbers is further preferably 0 or 1, particularly 0. The numbers q1 and q2 may be different from each other and are preferably the same. In a case where q1 denotes 2 or more, the species of the two or more R¹¹ may be the same or different from each other. The same relationship between q1 and R¹¹ also applies to q2 and R¹². The species of R¹¹ and R-2 may be the same or different from each other.

The substitution positions of R¹¹ and R¹² are not particularly limited to specific positions and are any positions other than the substitution positions of A⁵ and (poly)oxyalkylene groups [—O—(A⁶O)p1-] and [—O—(A O)p2-] in two naphthalene ring skeletons. Each substitution position of R¹¹ and R¹² is preferably any of 3 to 8-positions with respect to 1-position of the corresponding one of the two naphthalene ring skeletons bonded to AP. For example, in a case where A⁵ represents a direct bond (a single bond), R¹¹ are preferably bonded at any of 3-to 8-position and any of 3′- to 8′-position, respectively, of the 1, 1′-binaphthyl skeleton.

Examples of the alkylene group represented by A⁵ may include a C₁₋₄alkylene group such as methylene group, ethylene group, propylene group, trimethylene group, 1, 2-butanediyl group, and tetramethylene group. The preferred A⁵ is the direct bond or a C₁₋₂ alkylene group such as methylene group from the viewpoint of an optical characteristic such as a high refractive index, a low Abbe's number, and a low birefringence. In particular, the direct bond (the single bond) is preferred.

The alkylene groups A⁶ and A⁷ may be selected from the same as the alkylene groups A³ and A⁴ exemplified in the formula (2), including preferred aspects. The repeating numbers p1 and p2 may also selected from the same as the repeating numbers s1 and s2 in the formula (2), including preferred aspects.

In the formula (5), the bonding positions of the (poly)oxyalkylene groups [—O—(A°0)p1-] and [—O—(A⁷O)p2-] are not particularly limited to specific positions, and may for example be 2, 2′-positions or 4, 4′-positions with respect to the 1, 1′-binaphthyl skeleton. From the viewpoint of improved productivity due to easy preparation (synthesis) or supply and of easy achievement of a high refractive index from conformation relationship, the preferred bonding positions are 2, 2′-positions. Thus, the binaphthalene-containing diol represented by the formula (5) preferably includes a binaphthalene-containing diol represented by the following formula (5a):

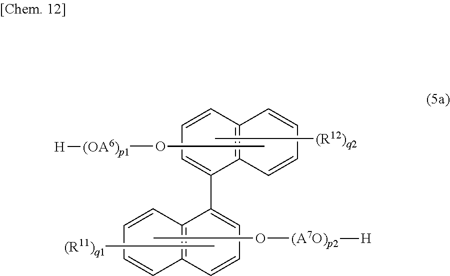

wherein A⁶, A⁷, p1, p2, R¹¹, R¹², q1, and q2 have the same meanings as defined in the formula (5).

Representative examples of the binaphthalene-containing diol represented by the formula (5a) may include a dihydroxy-1, 1′-binaphthyl such as 2,2′-dihydroxy-1, 1′-binaphthyl; and a bis[hydroxy(poly)alkoxy]-1, 1′-binaphthyl. Examples of the bis[hydroxy(poly)alkoxy]-1, 1′-binaphthyl may include a 2, 2′-bis[hydroxy (mono-to deca-) C₂₋₄alkoxy]-1, 1′-binaphthyl such as 2, 2′-bis(2-hydroxyethoxy)-1,1′-binaphthyl, 2,2′-bis(2-hydroxypropoxy)-1,1′-binaphthyl, and 2, 2′-bis[2-(2-hydroxyethoxy) ethoxy]-1, 1′-binaphthyl.

These binaphthalene-containing diols may be used alone or in combination of two or more. Among them, the diol represented by the formula (5a) is preferred, 2,2′-dihydroxy-1, 1′-binaphthyl or a 2, 2′-bis[hydroxy (mono-to tetra-) C₂₋₄alkoxy]-1, 1′-binaphthyl is further preferred, and a 2,2′-bis[hydroxy (mono-to tri-) C₂₋₃alkoxy]-1, 1′-binaphthyl such as 2, 2′-bis(2-hydroxyethoxy)-1,1′-binaphthyl is most preferred.

### (Other Diol Components)

The diol component may further contain another diol component. Another diol component is the same as another diol component (except for a binaphthol) of the fluorene-containing polyester-series resin, including preferred aspects.

### (Carbonate Ester Bond-Forming Component)

The fluorene-containing polycarbonate ester-series resin of the present disclosure contains a carbonate ester bond-forming component as a polymerization component for forming a carbonate ester unit, in addition to the diol component. The representative carbonate ester bond-forming component may include, for example, a phosgene such as phosgene and triphosgene, and a carbonate diester such as a diphenylcarbonate ester. From the viewpoint of safety or others, the carbonate diester such as the diphenylcarbonate ester is preferred,

### (Characteristics of Fluorene-Containing Polycarbonate Ester-Series Resin)

In the fluorene-containing polycarbonate ester-series resin of the present disclosure, the proportion of the fluorene-containing diol in the diol component may be not less than 10% by mol, and is preferably not less than 30% by mol, not less than 50% by mol, not less than 80% by mol, not less than 90% by mol, not less than 95% by mol, and 100% by mol in a stepwise manner. In the present disclosure, even the fluorene-containing polycarbonate ester-series resin having a high proportion of the fluorene skeleton, in particular, the fluorene-containing polycarbonate ester resin can efficiently depolymerized and allows efficient recovery of a useful fluorene skeleton-containing monomer component (or monomer).

In a case where the diol component further contains the binaphthalene-containing diol in addition to the fluorene-containing diol, the molar ratio of the fluorene-containing diol relative to the binaphthalene-containing diol may be selected from a range of about 99/1 to 10/90 in terms of the former/the latter, and a preferred range of the ratio is 97/3 to 20/80, 95/5 to 30/70, 90/10 to 35/65, 80/20 to 40/60, and 70/30 to 40/60 in a stepwise manner, and most preferably 60/40 to 40/60. The ratio of the both may be 93/7 to 40/60, preferably 90/10 to 50/50, and further preferably 80/20 to 60/40 in terms of the former/the latter.

In the fluorene-containing polycarbonate ester-series resin of the present disclosure, the total proportion of the fluorene-containing diol and the binaphthalene-containing diol in the diol component may be not less than 50% by mol, and is preferably not less than 60% by mol, not less than 70% by mol, not less than 80% by mol, not less than 90% by mol, not less than 95% by mol, and 100% by mol in a stepwise manner,

In the fluorene-containing polycarbonate ester-series resin of the present disclosure, the total proportion of the diol component and the carbonate ester bond-forming component in the whole polymerization component may be not less than 50% by mol, and is preferably not less than 60% by mol, not less than 70% by mol, not less than 808 by mol, not less than 90% by mol, not less than 95% by mol, and 100% by mol in a stepwise manner,

The weight-average molecular weight of the fluorene-containing polycarbonate ester-series resin is, for example, 20,000 to 100,000, preferably 25,000 to 80,000, further preferably 30,000 to 70,000, and most preferably 35,000 to 60,000.

In the description and the claims of this application, the weight-average molecular weight of the fluorene-containing polycarbonate ester-series resin can be measured by gel permeation chromatography in terms of a polystyrene as a standard material.

The fluorene-containing polycarbonate ester-series resin may be amorphous.

### (A) First Decomposition Step

The depolymerization method for the fluorene-containing polycarbonate ester-series resin of the present disclosure includes a first decomposition step of allowing the fluorene-containing polycarbonate ester-series resin to react with a carbonate ester in the presence of a first hydrolysis catalyst to decompose the fluorene-containing polycarbonate ester-series resin, obtaining a first decomposition product containing a monocarbonate ester form of a diol and/or a dicarbonate ester form of a diol.

### (Carbonate Ester)

The carbonate ester serves as a depolymerization agent of the fluorene-containing polycarbonate ester-series resin, The carbonate ester includes the same as the carbonate ester described in the fluorene-containing polyester-series resin, including preferred aspects.

The ratio of the carbonate ester relative to 1 mol of the fluorene-containing polycarbonate ester-series resin (the number of moles corresponding to number-average molecular weight) may be not less than 1 mol, and is, for example, 0.1 to 50 mol, preferably 0.5 to 30 mol, further preferably 1 to 10 mol, more preferably 1.5 to 5 mol, and most preferably 2 to 3 mol. The ratio of the carbonate ester relative to 100 parts by mass of the fluorene-containing polycarbonate ester-series resin may for example be not less than 5 parts by mass, and is, for example, 5 to 1000 parts by mass, preferably 10 to 100 parts by mass, further preferably 20 to 80 parts by mass, more preferably 30 to 70 parts by mass, and most preferably 40 to 60 parts by mass. In a case where the ratio of the carbonate ester is excessively low, the depolymerization may not proceed rapidly.

### (First Hydrolysis Catalyst)

The first hydrolysis catalyst may be selected from the same as the first hydrolysis catalyst for the fluorene-containing polyester-series resin, including preferred aspects. The first hydrolysis catalyst may be used alone or in combination of two or more.

The ratio of the first hydrolysis catalyst relative to 1 mol of the fluorene-containing polycarbonate ester-series resin may for example be not less than 0.01 mol, and is preferably 0.01 to 0.5 mol, further preferably 0.03 to 0.3 mol, more preferably 0.05 to 0.25 mol, and most preferably 0.1 to 0.2 mol. The ratio of the first hydrolysis catalyst relative to 100 parts by mass of the fluorene-containing polycarbonate ester-series resin may for example be not less than 0.1 parts by mass, and is preferably 0.1 to 10 parts by mass, further preferably 0.3 to 5 parts by mass, more preferably 0.5 to 4 parts by mass, and most preferably 1 to 3 parts by mass. In a case where the ratio of the hydrolysis catalyst is excessively low, the depolymerization may not proceed rapidly, The ratio of the first hydrolysis catalyst may be the ratio of the alkali catalyst.

### (Reaction Condition)

The reaction temperature may be not less than 20° C., and is, for example, 20 to 85° C., preferably 25 to 83° C., further preferably 30 to 80° C., more preferably 50 to 75° C., and most preferably 60 to 70° C.

The reaction time may be not less than 10 minutes, and is, for example, 10 minutes to 10 hours, preferably 30 minutes to 8 hours, and further preferably 1 to 5 hours.

The reaction may be carried out in the presence of an inert or inactive gas. From the viewpoint of convenience and others, the reaction is preferably carried out in the atmosphere, Moreover, the reaction may be carried out under a reduced pressure. From the viewpoint of convenience and others, the reaction is preferably carried out under the atmospheric pressure.

### (First Decomposition Product)

The first decomposition product in the first decomposition step contains a monocarbonate ester form of a diol and/or a dicarbonate ester form of a diol. Thus, the present disclosure includes a process for producing a monocarbonate ester form of a diol and/or a dicarbonate ester form of a diol by the first decomposition step, also for the fluorene-containing polycarbonate ester-series resin in the same manner as the fluorene-containing polyester-series resin.

The monocarbonate ester form of the diol and/or the dicarbonate ester form of the diol may be converted into a diol by subjecting to the next second decomposition step, and the resulting diol may be reused as a raw material for a fluorene-containing polycarbonate ester-series resin. The monocarbonate ester form of the diol and/or the dicarbonate ester form of the diol may be used as a carbonate ester compound without being subjected to the second decomposition step.

The monocarbonate ester form of the diol and the dicarbonate ester form of the diol may include the same as the monocarbonate ester form of the diol and the dicarbonate ester form of the diol for the fluorene-containing polyester-series resin, including preferred aspects.

The ratio of the monocarbonate ester form relative to 1 mol of the dicarbonate ester form is, for example, 0.1 to 30 mol, preferably 0.5 to 10 mol, further preferably 1 to 5 mol, and more preferably 2 to 4.

The first decomposition product may further contain a diol. The diol may be a diol corresponding to the monocarbonate ester form or the dicarbonate ester form.

In the first decomposition step, the total ratio of the monocarbonate ester form and the dicarbonate ester form relative to 1 mol of the diol in the first decomposition product is, for example, 0.1 to 5 mol, preferably 0.5 to 3 mol, further preferably 1 to 2 mol, and more preferably 1.2 to 1.7 mol.

In the first decomposition step, the total ratio of the monocarbonate ester form, the dicarbonate ester form, and the diol relative to 1 mol of the carbonate ester bond-forming component is, for example, 0.1 to 5 mol, preferably 0.2 to 3 mol, further preferably 0.3 to 2 mol, and more preferably 0.5 to 1.5 mol.

The first decomposition product may undergo a conventional purification treatment. The conventional purification treatment may include, for example, optionally subjecting a mixture containing the decomposition product to neutralization and washing with water, and then removing impurities from the mixture by filtration or other means. In a case where the monomer component in the mixture is precipitated, the monomer component may completely be dissolved using a good solvent for the monomer component and then the resulting mixture may be subjected to neutralization and washing with water.

In the first decomposition step, the monocarbonate ester form of the diol and/or the dicarbonate ester form of the diol is mainly obtained. Thus, in a case where the novel carbonate ester compound is an objective compound, the depolymerization method of the present disclosure may be a depolymerization method of which decomposition step only contains the first decomposition step and does not contain the after-mentioned second decomposition step.

### (B) Second Decomposition Step

The depolymerization method for the fluorene-containing polycarbonate ester-series resin of the present disclosure may have a second decomposition step for obtaining a second decomposition product, in addition to the first decomposition step. In the second decomposition step, the first decomposition product is allowed to react with an alcohol to obtain a diol. Specifically, in the second decomposition step, the monocarbonate ester form of the diol and/or the dicarbonate ester form of the diol is allowed to react with the alcohol to obtain a diol component. Thus, since the second decomposition step allows the monocarbonate ester form of the diol and/or the dicarbonate ester form of the diol to be converted into a diol, the second decomposition step is particularly effective in recovering a fluorene skeleton-containing diol component as a monomer component.

### (Alcohol)

The alcohol may be selected from the same as the alcohol in the second decomposition step for the fluorene-containing polyester-series resin, including preferred aspects. The alcohol may be used alone or in combination of two or more.

The ratio of the alcohol relative to 100 parts by mass of the first decomposition product may be not less than 50 parts by mass, and is, for example, 50 to 1000 parts by mass, preferably 80 to 500 parts by mass, further preferably 100 to 300 parts by mass, and more preferably 120 to 200 parts by mass. In a case where the ratio of the alcohol is excessively low, the yield of the diol component may be reduced.

### (Second Hydrolysis Catalyst)

Examples of a second hydrolysis catalyst may include the hydrolysis catalyst exemplified as the first hydrolysis catalyst for the fluorene-containing polyester-series resin. The second hydrolysis catalyst may be used alone or in combination of two or more. Among the second hydrolysis catalysts, the alkali metal hydroxide or the alkaline earth metal hydroxide is preferred, and the alkali metal hydroxide such as potassium hydroxide is particularly preferred.

The ratio of the second hydrolysis catalyst relative to 100 parts by mass of the first decomposition product may be not less than 1 part by mass, and is, for example, 1 to 30 parts by mass, preferably 5 to 20 parts by mass, and further preferably 5 to 10 parts by mass. In a case where the ratio of the alkali catalyst is excessively low, the yield of the diol component may be reduced. The ratio of the second hydrolysis catalyst may be the ratio of the alkali catalyst.

### (Reaction Condition)

The reaction temperature may be not less than 0° C., and is, for example, 5 to 80° C., preferably 10 to 50° C., and further preferably 15 to 40° C. The reaction temperature may be a room temperature.

The reaction time may be not less than 5 minutes, and is, for example, 5 minutes to 5 hours, preferably 10 minutes to 3 hours, further preferably 30 minutes to 2 hours, and more preferably 40 minutes to 1.5 hours.

The reaction may be carried out in the presence of an inert or inactive gas. From the viewpoint of convenience and others, the reaction is preferably carried out in the atmosphere. Moreover, the reaction may be carried out under a reduced pressure. From the viewpoint of convenience and others, the reaction is preferably carried out under the atmospheric pressure.

### (Second Decomposition Product)

The second decomposition product in the second decomposition step contains a diol. Specifically, in a case where the depolymerization method of the present disclosure includes the second decomposition step, raw materials of the fluorene-containing resin can be recovered. Thus, the resulting monomer components can be used as they are to newly synthesize a fluorene-containing resin, improving the recyclability of the fluorene-containing resin. The fluorene-containing resin may be a fluorene-containing polyester-series resin or may be a fluorene-containing polycarbonate ester-series resin.

### (C) Purification Step

The second decomposition product containing the diol obtained in the second decomposition step may undergo a conventional purification treatment. The conventional purification treatment may include, for example, optionally subjecting a mixture containing the second decomposition product to neutralization and washing with water, then removing impurities from the mixture by filtration or others, and then subjecting the resulting product to crystallization.

As the crystallization method, it is preferred to crystallize the monomer component with a crystallization solvent containing an aromatic hydrocarbon.

As the aromatic hydrocarbon, preferred is a mono- or di-C₁₋₂alkyl-benzene such as toluene, xylene, and ethylbenzene, and particularly preferred is toluene.

The ratio of the crystallization solvent relative to 100 parts by mass of the diol is, for example, 10 to 3000 parts by mass, preferably 50 to 2000 parts by mass, further preferably 100 to 1000 parts by mass, and most preferably 200 to 500 parts by mass.

In the crystallization treatment, dissolution of the diol in the crystallization solvent and cooling of the resulting solution allows precipitation or crystallization of the diol having a higher purity. The temperature for dissolving the diol in the crystallization solvent is a temperature lower than the boiling point of the solvent and is, for example, 30 to 200° C., preferably 50 to 150° C., and further preferably 60 to 100° C. By a conventional method, the crystallization-treated diol may be washed and then dried,

### [Recycling Method of Fluorene-Containing Resin]

The decomposition product obtained according to the depolymerization method of the present disclosure can be used as a monomeric raw material for a fluorene-containing resin with a fluorene skeleton and an ester bond and/or a carbonate ester bond to obtain a newly produced fluorene-containing resin, and thus the decomposition product can be recycled. Specifically, the recycling method of the present disclosure includes a depolymerization step of allowing the fluorene-containing resin to react with a carbonate ester to obtain a decomposition product, and a polymerization step (a repolymerization step) of polymerizing the decomposition product obtained in the depolymerization step to obtain a newly produced fluorene-containing resin. The decomposition product may be the first decomposition product or may be the second decomposition product, and is preferably the second decomposition product.

In the recycling method of the present disclosure, the decomposition product obtained in the depolymerization step may be purified and the purified product may be subjected to the polymerization step, or the decomposition product may be subjected to the polymerization step without purification. Moreover, in the polymerization step, a new monomeric raw material may be supplemented to the monomeric raw material as the decomposition product. The monomeric raw material to be newly supplemented may be an insufficient monomeric raw material depending on the composition of the decomposition product.

In the polymerization step, as the method for polymerizing the fluorene-containing resin using the decomposition product, there may be used a conventional method according to the species of the fluorene-containing resin. As the method for polymerizing the fluorene-containing polyester-series resin, there may be used, for example, methods described in Japanese Patent Application Laid-Open Publication Nos. 2013-064117, 2013-064118, 2014-218645 and 2016-069643, and Japanese Patent No. 7016976. The method for polymerizing the fluorene-containing polycarbonate ester-series resin may include, for example, methods described in Japanese Patent Application Laid-Open Publication Nos. 2018-104691 and 2021-134216.

### EXAMPLES

The following examples are intended to describe this disclosure in further detail and should by no means be interpreted as defining the scope of the disclosure. The details of materials used in Examples and each evaluation method are shown below.

### [Material]

FDP-m: 9, 9-Bis (2-methoxycarbonylethyl) fluorene represented by the following formula (synthesized in the same manner as Example 1 of Japanese Patent Application Laid-Open Publication No. 2005-89422 except that methyl acrylate [37, 9 g (0.44 mol)] was used instead of t-butyl acrylate.)

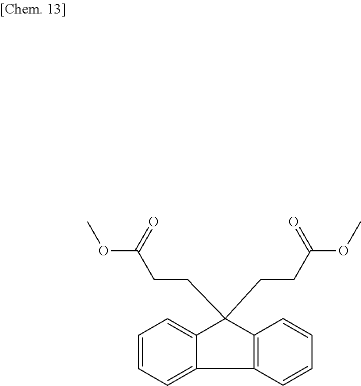

DNEDP-m: 9, 9-Bis (2-methoxycarbonylethyl)-2, 7-di(2-naphthyl) fluorene represented by the following formula (synthesized according to Example 1A of WO 2020/213470.)

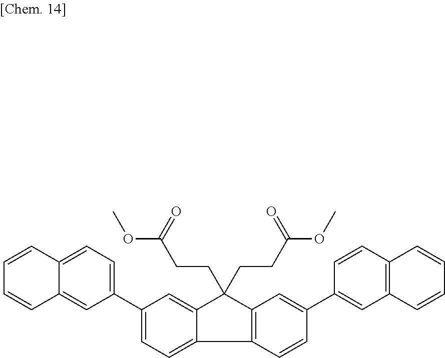 [SMILES: COC(=O)CCC1(CCC(=O)OC)c2cc(-c3ccc4ccccc4c3)ccc2-c2ccc(-c3ccc4ccccc4c3)cc21]

DMT: Dimethyl terephthalate
DMN: Dimethyl 2, 6-naphthalenedicarboxylate
BPEF: 9, 9-Bis[4-(2-hydroxyethoxy)phenyl]fluorene, manufactured by Osaka Gas Chemicals Co., Ltd.
BNEF: 9, 9-Bis[6-(2-hydroxyethoxy)-2-naphthyl]fluorene, manufactured by Osaka Gas Chemicals Co., Ltd.
BOPPEF: 9, 9-Bis[4-(2-hydroxyethoxy)-3-phenylphenyl]fluorene, manufactured by Osaka Gas Chemicals Co., Ltd.
EG: Ethylene glycol
BINOL-2EO: 2, 2′-Bis (2-hydroxyethoxy)-1,1′-binaphthyl (synthesized in accordance with Synthesis Example 2 described in Japanese Patent Application Laid-Open Publication No. 2018-59074.)
Dimethyl carbonate: manufactured by Tokyo Chemical Industry Co., Ltd.
LiOMe: Lithium methoxide, manufactured by Tokyo Chemical Industry Co., Ltd.
NaOMe: Sodium methoxide, manufactured by FUJIFILM Wako Pure Chemical Corporation

### [LC-MS Production Ratio]

Used were “Nexera XR” as an HPLC (high-performance or high-speed liquid chromatograph) apparatus and “LCMS-2020” as an MS unit, each manufactured by SHIMADZU CORPORATION, and “Kinetex C-18” manufactured by Phenomenex as a column. A sample was dissolved in acetonitrile and subjected to measurement, and a LC-MS production ratio was calculated from a ratio of % by area in HPLC.

### [Yield]

A diol component and a dicarboxylic acid component in a polyester resin were supposed as 50:50 (molar ratio) in terms of the former: the latter. A yield was calculated from a blending ratio of each monomer component and a molecular weight of each monomer. Moreover, a diol component and a carbonate ester bond-forming component in a polycarbonate ester-series resin were supposed as 50:50 (molar ratio) in terms of the former: the latter. A yield was calculated from a blending ratio of each monomer component and a molecular weight of each monomer.

### [LC Purity]

A purity [% by area] was calculated using the above-described HPLC apparatus.

### [Glass Transition Temperature (Tg)]

A glass transition temperature was measured under a nitrogen atmosphere at a heating rate of 10° C./minute using a differential scanning calorimeter (“DISCOVERY DSC25” manufactured by TA Instruments),

### [Molecular Weight Distribution]

Used were “HLC-8320GPC” manufactured by Tosoh Corporation as a GPC (gel permeation chromatography) apparatus and “TSKgel” manufactured by Tosoh Corporation as a column. A sample was dissolved in THF and subjected to measurement, and a molecular weight was calculated in terms of polystyrene.

### [Refractive Index]

A sample was heat-pressed at 200 to 240° C. to form a film having a thickness of 200 to 300 μm. This film was cut into a strip shape with a length of 20 to 30 mm and a width of 10 mm to obtain a test piece. The refractive index nD of the resulting test piece at 589 nm (D-line) was measured at a measurement temperature of 20° C. using a multi-wavelength Abbe refractometer (“DR-M4 (circulating constant-temperature water bath 60-C₃)” manufactured by ATAGO CO., LTD.) and diiodomethane as a contact liquid.

### [Abbe's Number]

Using the test piece of which the refractive index nD at 589 nm (D-line) was measured, the refractive indexes nF and nC were measured in the same manner as in the refractive index nD except that the measurement wavelength was changed to 486 nm (F-line) and 656 nm (C-line), respectively. From the resulting refractive indexes nF, nD, and nC at the respective wavelengths, the Abbe's number was calculated by the following formula:

$$( Abbe ’ s number ) = ( nD - 1 ) / ( nF - nC )$$

### [Birefringence (Three Times Stretching)]

A sample was heat-pressed at 200 to 240° C. to form a film having a thickness of 200 to 600 μm. This film was cut into a strip shape with a length of 10 mm and a width of 50 mm, and the strip-shaped film was uniaxially stretched so that the stretching ratio was 3 at 25 mm/minute under a temperature condition of the glass transition temperature Tg+10° C., thus obtaining a test piece. For the obtained test piece, the retardation was measured by a parallel Nicol rotation method using a retardation film/optical material inspection apparatus (“RETS-100” manufactured by Otsuka Electronics Co., Ltd.) under the conditions of a measurement temperature of 20° C. and a measurement wavelength of 600 nm, the resulting value was divided by the thickness of the measurement site to calculate a birefringence (or a three times birefringence).

### Example 1

### (Preparation of Polymer A)

Following a conventional method, in accordance with Production Example 2 in Examples of Japanese Patent No. 7016976, Polymer A was prepared in which a constitutional unit derived from DNFDP-m was 100% by mol in a dicarboxylic acid unit, and a constitutional unit derived from BPEF was 70% by mol and a constitutional unit derived from EG was 30% by mol in a diol unit.

### (First Decomposition Step)

The Polymer A was mixer-ground, and to 45.82 g of the mixer-ground Polymer A (the total amount of monomers incorporated in the Polymer A corresponds to about 0.1 mol) were added 90.1 g of dimethyl carbonate and 4.58 g of a methanol solution containing LiOMe at a concentration of 10% by mass, and the mixture underwent a reaction at 65° C. for 3 hours. To the resulting reaction mixture were added 18 g of water and 3.5 g of a 10% by mass HCl aqueous solution, and the mixture was stirred at 65° C. for 15 minutes. Then, the separated organic phase was recovered and was filtered to remove insoluble matter. The resulting organic phase was analyzed by

LC-MS with the following results.

### BPEF: 1.880% by Area

BPEF monocarbonate ester form represented by the following formula: 11.265% by area

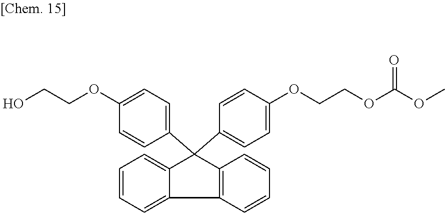 [SMILES: COC(=O)OCCOc1ccc(C2(c3ccc(OCCO)cc3)c3ccccc3-c3ccccc32)cc1]

BPEF dicarbonate ester form represented by the following formula: 18.499% by area

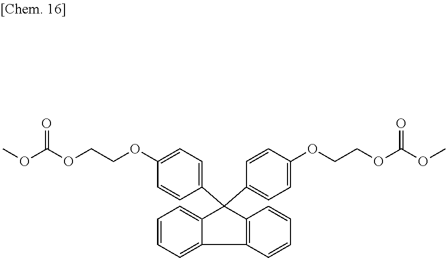

### DNFDP-m: 51.131% by Area

### (Second Decomposition Step)

The organic phase was concentrated to obtain a crude product. To the crude product were added 80.1 g of methanol and 7.0 g of potassium hydroxide, and the mixture underwent a reaction for one hour under a reflux condition at 65° C. The reaction mixture was filtered at a temperature of not lower than 60° C. and was washed with 150 g of methanol at 40° C. to obtain 30.1 g of crude DNFDP-m crystal as a filtration residue. The filtrate obtained by this filtration contains BPEF as a main component.

### (Purification of DNFDP-m)

To 30.1 g of the crude crystal was added 75.1 g of toluene, and the mixture was completely dissolved at 80° C. Then, to the solution was added 20 g of water and 5 g of a 10% by mass HCl aqueous solution, and the resulting mixture was stirred at 80° C. for 15 minutes. The mixture was allowed to stand, the separated aqueous phase was removed, then the organic phase was washed with 20 g of water, and this washing operation was conducted three times. The organic phase at 80° C. was allowed to cool to a room temperature and was stirred at a room temperature for one hour, and then was filtered. The resulting residue was washed with toluene and methanol in a sequential order. The washed filtration residue was dried under a reduced pressure at 80° C. for 12 hours to obtain 14.7 g of a white solid matter (or a white solid) containing DNFDP-m (yield 50%, LC purity 97.4%),

### (Purification of BPEF)

The filtrate containing BPEF as a main component was stirred at 5 to 10° C. for one hour, and then was filtered and was washed with methanol at 10° C. to obtain 15.1 g of a crude crystal. To the resulting crude crystal was added 52.8 g of toluene, and the mixture was completely dissolved at 80° C. Then, to the solution were added 20 g of water and 5 g of a 10% by mass HCl aqueous solution, and the resulting mixture was stirred at 80° C. for 15 minutes. The mixture was allowed to stand, the separated aqueous phase was removed, then the organic phase was washed with 20 g of water, and this washing operation was conducted three times. The organic phase at 80° C. was allowed to cool to a room temperature and was stirred at 5 to 10° C. for one hour, and then was filtered. The resulting residue was washed with toluene at 10° C. The washed filtration residue was dried under a reduced pressure at 80° C. for 12 hours to obtain 10.7 g of a white solid matter containing BREF (yield 70%, LC purity 98.9%).

### Example 2

### (Preparation of Polymer B)

Following a conventional method, in accordance with Production Example 2 in Examples of Japanese Patent No. 7016976, Polymer B was prepared in which a constitutional unit derived from DNFDP-m was 100% by mol in a dicarboxylic acid unit, and a constitutional unit derived from BPEF was 10% by mol and a constitutional unit derived from EG was 90% by mol in a diol unit.

### (First Decomposition Step)

The Polymer B was mixer-ground, and to 3.45 g of the mixer-ground Polymer B (the total amount of monomers incorporated in the Polymer B corresponds to about 0.01 mol) were added 9.01 g of dimethyl carbonate and 0.35 g of a methanol solution containing LiOMe at a concentration of 10% by mass, and the mixture underwent a reaction at 65° C. for 3 hours. To the resulting reaction mixture were added g of toluene, and the mixture was completely 10 dissolved at 65° C. Then, to the solution were added 3.0 g of water and 0.34 g of a 10% by mass HCl aqueous solution, and the mixture was stirred at 65° C. for 15 minutes. The separated aqueous phase was removed, the organic phase washed with water, and this washing operation was conducted three times. Then, the washed organic phase was filtered to remove insoluble matter and was concentrated to obtain a crude product.

### (Second Decomposition Step)

To the crude product were added 6.4 g of methanol and 0.56 g of potassium hydroxide, and the mixture underwent a reaction for one hour under a reflux condition at 65° C. The reaction mixture was filtered at 50° C. and was washed with 15 g of methanol at 40° C. to obtain 3.35 g of crude DNFDP-m crystal as a filtration residue. The filtrate obtained by this filtration contains BPEF as a main component.

### (Purification of DNFDP-m)

To 3.35 g of the crude crystal was added 8.13 g of toluene, and the mixture was completely dissolved at 80° C. Then, to the solution was added 3.0 g of water and 0.6 g of a 10% by mass HCl aqueous solution, and the resulting mixture was stirred at 80° C. for 15 minutes. The mixture was allowed to stand, the separated aqueous phase was removed, then the organic phase was washed with 3.0 g of water, and this washing operation was repeated three times, The organic phase at 80° C. was allowed to cool to a room temperature and was stirred at 5 to 10° C. for one hour. The organic phase was filtered, and the resulting residue was washed with methanol, and then the washed filtration residue was dried under a reduced pressure at 50° C. for 12 hours to obtain 2.48 g of a white solid matter containing DNFDP-m (yield 84%, LC purity 93.7%).

### (Purification of BPEF)

To the resulting filtrate B were added 0.6 g of a 10% by mass HCl aqueous solution and 5 g of water. The mixture was stirred at 5 to 10° C. for one hour and then was filtered, and the resulting residue was washed with methanol at 10° C. to obtain 0.09 g of a crude crystal. To the resulting crude crystal was added 2.25 g of toluene, and the mixture was completely dissolved at 80° C. Then, the solution was allowed to cool and was stirred at 5 to 10° C. for one hour. The stirred mixture was filtered, the filtration residue was washed with toluene at 10° C., and then the washed filtration residue was dried under a reduced pressure at 50° C. for 12 hours to obtain 0.04 g of a white solid matter containing BPEF (yield 18%, IC purity 94.2%).

### Example 3

### (Preparation of Polymer C)

Following a conventional method, in accordance with Example 16 of Japanese Patent Application Laid-Open publication No. 2016-069643, Polymer C was prepared in which a constitutional unit derived from FDP-m was 100% by mol in a dicarboxylic acid unit, and a constitutional unit derived from BNEF was 70% by mol and a constitutional unit derived from EG was 30% by mol in a diol unit.

### (First Decomposition Step)

The Polymer C was mixer-ground, and to 3.67 g of the mixer-ground Polymer C (the total amount of monomers incorporated in the Polymer C corresponds to about 0.01 mol) were added 9.01 g of dimethyl carbonate and 0.37 g of a methanol solution containing LiOMe at a concentration of 10% by mass, and the mixture underwent a reaction at 65° C. for 2 hours. To the resulting reaction mixture were added 0.9 g of water and 0.37 g of a 10% by mass HCl aqueous solution, and the mixture was stirred at 65° C. for 15 minutes. Then, the separated organic phase was recovered and was filtered to remove insoluble matter. The resulting organic phase was analyzed by LC-MS with the following results.

### BNEF: 4.400% by Area

BNEF monocarbonate ester form represented by the following formula: 22.806% by area

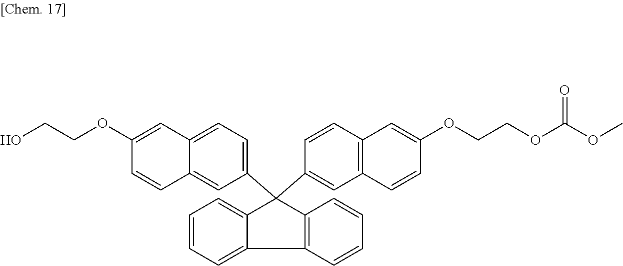 [SMILES: COC(=O)OCCOc1ccc2cc(C3(c4ccc5cc(OCCO)ccc5c4)c4ccccc4-c4ccccc43)ccc2c1]

BNEF dicarbonate ester form represented by the following formula: 37.648% by area

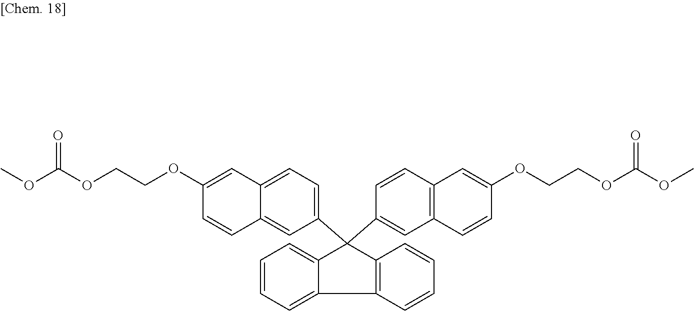

FDP-m: 18.616% by area.

### (Second Decomposition Step)

The organic phase was concentrated to obtain a crude product. To the crude product were added 3.2 g of methanol and 0.56 g of potassium hydroxide, and the mixture underwent a reaction for one hour under a reflux condition at 65° C. After the reaction was completed, to the reaction mixture was added 9.6 g of heptane, and the resulting mixture was stirred at 65° C. for 10 minutes. Thereafter, the resulting lower phase (containing methanol and BNEF) and upper phase (containing heptane and FDP-m) were recovered separately.

### (Purification of FDP-m)

The above-described upper phase was concentrated, and the concentrate was dried under a reduced pressure at 50° C. for 12 hours to obtain 0.13 g of a white solid matter containing FDP-m (yield 8%, LC purity 92.2%),

### (Purification of BNEF)

To the above-described lower phase were added 10 g of toluene, 4 g of a 10% by mass HCl aqueous solution, and 3 g of water. The mixture was stirred at 80° C., and then was allowed to cool to a room temperature and was stirred at a room temperature for one hour. Thereafter, the mixture was filtered, and the resulting residue was washed with 2-propanol. The washed filtration residue was dried under a reduced pressure at 50° C. for 12 hours to obtain 0.85 g of a white solid matter containing BNEF (yield 45%, LC purity 96.0%).

### Example 4

### (Preparation of Polymer D)

Following a conventional method, in accordance with Reference Example 6 of Japanese Patent Application Laid-Open Publication No. 2013-064117, Polymer D was prepared in which a constitutional unit derived from DMT was 100% by mol in a dicarboxylic acid unit, and a constitutional unit derived from BPEF was 70% by mol and a constitutional unit derived from EG was 30% by mol in a diol unit.

### (First Decomposition Step)

The Polymer D was mixer-ground, and to 2. 60 g of the mixer-ground Polymer D (the total amount of monomers incorporated in the Polymer D corresponds to about 0.01 mol) were added 9.01 g of dimethyl carbonate and 0.26 g of a methanol solution containing LiOMe at a concentration of 10% by mass, and the mixture underwent a reaction at 65° C. for 3 hours. To the resulting reaction mixture were added 0.9 g of water, 0.26 g of a 10% by mass HCl aqueous solution, and 5 g of ethyl acetate, and the mixture was stirred at 65° C. for 15 minutes. Then, the separated organic phase was recovered and was filtered to remove insoluble matter.

### (Second Decomposition Step)

The organic phase was concentrated to obtain a crude product. To the crude product were added 9,6 g of methanol and 0.56 g of potassium hydroxide, and the mixture underwent a reaction for one hour under a reflux condition at 65° C. After the reaction was completed, to the reaction mixture was added 3 g of a 10% by mass HCl aqueous solution, and the resulting mixture was stirred at 5 to 10° C. for 30 minutes.

### (Purification of BPEF)

The stirred mixture was filtered, and the resulting residue was washed with methanol. The washed filtration residue was dried under a reduced pressure at 80° C. for 12 hours to obtain 1.5 g of a white solid matter containing BPEF (yield 98%, LC purity 93.6%).

### Example 5

### (Preparation of Polymer E)

Following a conventional method, in accordance with Example 3 of Japanese Patent Application Laid-Open Publication No. 2014-218645, Polymer E was prepared in which a constitutional unit derived from FDP-m was 100% by mol in a dicarboxylic acid unit, and a constitutional unit derived from BPEF was 80% by mol and a constitutional unit derived from EG was 20% by mol in a diol unit.

### (First Decomposition Step)

The Polymer E was mixer-ground, and to 3.50 g of the mixer-ground Polymer E (the total amount of monomers incorporated in the Polymer E corresponds to about 0.01 mol) were added 9.01 g of dimethyl carbonate and 0,35 g of a methanol solution containing LiOMe at a concentration of 10% by mass, and the mixture underwent a reaction at 65° C. for 3 hours. To the resulting reaction mixture were added 0.9 g of water and 0.37 g of a 10% by mass HCl aqueous solution, and the mixture was stirred at 65° C. for 15 minutes. Then, the separated organic phase was recovered and was filtered to remove insoluble matter.

### (Second Decomposition Step)

The organic phase was concentrated to obtain a crude product. To the crude product were added 3.2 g of methanol and 0.56 g of potassium hydroxide, and the mixture underwent a reaction for one hour under a reflux condition at 65° C. After the reaction was completed, to the reaction mixture was added 9.6 g of heptane, and the resulting mixture was stirred at 65° C. for 10 minutes. Thereafter, the resulting lower phase (containing methanol and BPEF) and upper phase (containing heptane and FDP-m) were recovered separately.

(Purification of FDP-m) The above-described upper phase was concentrated, and the concentrate was dried under a reduced pressure at 50° C. for 12 hours to obtain 0.10 g of a white solid matter containing FDP-m (yield 6%, LC purity 94.9%).

### (Purification of BPEF)

To the above-described lower phase were added 8 g of toluene and 3 g of a 10% by mass HCl aqueous solution. The mixture was stirred at 60° C., and then was allowed to slowly cool to a room temperature and was stirred at a room temperature for one hour, Thereafter, the mixture was filtered, and the resulting residue was washed with methanol to obtain 1.63 g of a crude crystal. To the crude crystal was added 6.52 g of toluene, and the mixture was dissolved at 85° C. Then, the solution was allowed to cool to a room temperature for crystallization. The filtration was conducted, and the residue was washed with methanol, and the washed residue was dried under a reduced pressure at 50° C. for 12 hours to obtain 0.31 g of a white solid matter containing BPEF (yield 18%, LC purity 94.4%).

### Example 6

### (First Decomposition Step)

The Polymer A was mixer-ground, and to 4.582 g of the mixer-ground Polymer A (the total amount of monomers incorporated in the Polymer A corresponds to about 0.01 mol) were added 9.01 g of dimethyl carbonate and 0.916 g of a methanol solution containing NaOMe at a concentration of 10% by mass, and the mixture underwent a reaction at 65° C. for 2 hours. To the resulting reaction mixture were added 1.5 g of water and 0.55 g of a 10% by mass HCl aqueous solution, and the mixture was stirred at 65° C. for 15 minutes. Then, the separated organic phase was recovered and was filtered to remove insoluble matter.

### (Second Decomposition Step)

The organic phase was concentrated to obtain a crude product. To the crude product were added 8.01 g of methanol and 0.56 g of potassium hydroxide, and the mixture underwent a reaction for one hour under a reflux condition at 65° C. The reaction mixture was filtered while still maintaining a temperature of not lower than 60° C., and the residue was washed with 15.0 g of methanol at 40° C. to obtain 4.4 g of crude DNFDP-m crystal. The filtrate obtained by this filtration contains BPEF as a main component.

### (Purification of DNEDP-m)

To 4.4 g of the crude crystal was added 10.9 g of toluene, and the mixture was completely dissolved at 80° C. Then, to the solution was added 5 g of water and 0.5 g of a 10% by mass HCl aqueous solution, and the resulting mixture was stirred at 80° C. for 15 minutes. The mixture was allowed to stand, the separated aqueous phase was removed, then the organic phase was washed with 5 g of water, and this washing operation was conducted three times. The organic phase at 80° C. was allowed to cool to a room temperature and was stirred at 5 to 10° C. for one hour, and then was filtered. The resulting residue was washed with toluene and methanol in a sequential order. The washed filtration residue was dried under a reduced pressure at 50° C. for 12 hours to obtain 1.56 g of a white solid matter containing DNFDP-m (yield 53%, LC purity 96.4%),

### (Purification of BPEF)

The filtrate F was stirred at 5 to 10° C. for one hour, and then was filtered. The residue was washed with methanol at 10° C. to obtain 1.03 g of a crude crystal. To the resulting crude crystal was added 3.08 g of toluene, and the mixture was completely dissolved at 80° C. Then, to the solution were added 1 g of water and 0.5 g of a 10% by mass HCl aqueous solution, and the resulting mixture was stirred at 80° C. for 15 minutes. The mixture was allowed to stand, the separated aqueous phase was removed, then the organic phase was washed with 1 g of water, and this washing operation was conducted three times. The organic phase at 80° C. was allowed to cool to a room temperature and was stirred at 5 to 10° C. for one hour, and then was filtered. The resulting residue was washed with methanol at 10° C. The washed filtration residue was dried under a reduced pressure at 50° C. for 12 hours to obtain 0.45 g of a white solid matter containing BREF (yield 29%, LC purity 98.1%).

### Example 7

### (Preparation of Polymer F)

Following a conventional method, in accordance with Example 7 of Japanese Patent Application Laid-Open Publication No. 2013-064118, Polymer E was prepared in which a constitutional unit derived from FDP-m was 100% by mol in a dicarboxylic acid unit, and a constitutional unit derived from BOPPEF was 70% by mol and a constitutional unit derived from EG was 30% by mol in a diol unit.

### (First Decomposition Step)

The Polymer F was mixer-ground, and to 4.12 g of the mixer-ground Polymer F (the total amount of monomers incorporated in the Polymer F corresponds to about 0.01 mol) were added 9.01 g of dimethyl carbonate and 0.41 g of a methanol solution containing LiOMe at a concentration of 10% by mass, and the mixture underwent a reaction at 65° C. for 4 hours. To the resulting reaction mixture were added 0.9 g of water and 0.41 g of a 10% by mass HCl aqueous solution, and the mixture was stirred at 65° C. for 15 minutes. Then, the separated organic phase was recovered and was filtered to remove insoluble matter.

### (Second Decomposition Step)

The organic phase was concentrated to obtain a crude product. To the crude product were added 11.2 g of methanol and 0.56 g of potassium hydroxide, and the mixture underwent a reaction for one hour under a reflux condition at 65° C. After the reaction was completed, the reaction mixture was allowed to slowly cool to a room temperature, was filtered, and was washed with 15.0 g of methanol to obtain 3.3 g of crude BOPPEF crystal. The filtrate obtained by this filtration contains FDP-m as a main component.

### (Purification of FDP-m)

To the above-described filtrate containing FDP-m as a main component was added 25 g of heptane, and the mixture was stirred at 40° C. for one hour. Thereafter, the resulting upper phase was concentrated, and the concentrate was dried under a reduced pressure at 50° C. for 12 hours to obtain 0.21 g of a white solid matter containing FDP-m (yield 12%, LC purity 96.3%).

### (Purification of BOPPEF)

To the above-described crude crystal were added 11.4 g of toluene, 5 g of water, and 0.2 g of a 10% by mass HCl aqueous solution, and the mixture was stirred at 60° C. Thereafter, an operation of washing the mixture with 5 g of water was conducted, and the washed mixture was allowed to slowly cool to a room temperature for crystallization. The filtration was conducted, and the residue was washed with methanol.

Thereafter, the washed residue was dried under a reduced pressure at 80° C. for 12 hours to obtain 1.99 g of a white solid matter containing BOPPEF (yield 84%, LC purity 97.2%).

For Examples 1 to 7, Table 1 shows the composition of the raw material polymer, the composition of the product after the first decomposition step, and the yields and purities of the dicarboxylic acid ester and diol after the purification step following the second decomposition step.

TABLE 1

| | | | | | | | | First decomposition step product | | | | Yield (% by mol) and | | | |
|---|---|---|---|---|---|---|---|---|---|---|---|---|---|---|---|
| | | | | | | | | LC-MS composition ratio (% by area) | | | | LC purity (% by area) | | | |
| | Composition of raw material polymer (molar ratio) | | | | | | | Dicar- | Mono- | Dicar- | | Dicarboxylic | | | |
| | Dicarboxylic acid unit | | | Diol unit | | | | boxylic | carbonate | bonate | | acid ester | | Diol | |
| | FDP-m | DNFDP-m | DMT | BPEF | BNEF | BOPPEF | EG | acid ester | ester form | ester form | Diol | Yield | Purity | Yield | Purity |
| Example 1 | — | 1 | — | 0.7 | — | — | 0.3 | 51.131 | 11.265 | 18.499 | 1.880 | 50 | 97.4 | 70 | 98.9 |
| Example 2 | — | 1 | — | 0.1 | — | — | 0.9 | 84.871 | 3.741 | 4.408 | 0.711 | 84 | 93.7 | 18 | 94.2 |
| Example 3 | 1 | — | — | — | 0.7 | — | 0.3 | 18.616 | 22.806 | 37.648 | 4.400 | 8 | 92.2 | 45 | 96.0 |
| Example 4 | — | — | 1 | 0.7 | — | — | 0.3 | 44.075 | 13.036 | 23.334 | 2.251 | — | — | 98 | 93.6 |
| Example 5 | 1 | — | — | 0.8 | — | — | 0.2 | 41.904 | 17.517 | 26.103 | 2.394 | 6 | 94.9 | 18 | 94.4 |
| Example 6 | — | 1 | — | 0.7 | — | — | 0.3 | 52.024 | 17.469 | 10.348 | 7.295 | 53 | 96.4 | 29 | 98.1 |
| Example 7 | 1 | — | — | — | — | 0.7 | 0.3 | 25.187 | 23.400 | 34.761 | 4.148 | 12 | 96.3 | 84 | 97.2 |

As apparent from the results of Table 1, all Examples 1 to 7 produce novel monocarbonate ester form and dicarbonate ester in the first decomposition step and further enabled recovery of the dicarboxylic acid ester and the diol through the second decomposition step and the purification step.

### Example 8

### (Preparation of Polymer G)

Following a conventional method, in accordance with Example 6 of Japanese Patent Application Laid-Open Publication No. 2013-064118, Polymer G was prepared in which a constitutional unit derived from DMN was 30% by mol and a constitutional unit derived from FDP-m was 70% by mol in a dicarboxylic acid unit, and a constitutional unit derived from BPEF was 85% by mol and a constitutional unit derived from EG was 15% by mol in a diol unit (Polymer G having a composition ratio in Table 2).

### (Depolymerization of Polyester G)

To 346.10 g of the pellet Polymer G (the total amount of monomers incorporated in the Polymer G corresponds to about 1.0 mol) were added 630.56 g of dimethyl carbonate and 34. 610 g of a methanol solution containing LiOMe at a concentration of 10% by mass, and the mixture underwent a reaction at 65° C. for 3 hours. To the resulting reaction mixture were added 250 g of water, 34 g of a 10% by mass HCl aqueous solution, and 600 g of toluene, and the mixture was stirred at 65° C. Thereafter, the resulting aqueous phase was removed. An operation of washing the residual organic phase with water was conducted until the resulting aqueous phase had a conductivity of not more than 10 μS/cm, and then the resulting organic phase was filtered to remove insoluble matter. The organic phase was concentrated to obtain a crude product. To the crude product were added 480 g of methanol and 50.5 g of potassium hydroxide, and the mixture underwent a reaction at a room temperature for 2 hours. To the reaction mixture were added 100 g of a 10% by mass HCl aqueous solution and 300 g of water, and the mixture was stirred at a room temperature for one hour. The stirred mixture was suction-filtered, and the residue was washed with warm water at 60° C. until the filtrate had a conductivity of not more than 10 μs/cm. Thereafter, the washed residue was dried under a reduced pressure at 50° C. to obtain 291 g of a white solid matter (calculation of molar ratio from HPLC measurement: BPEF/DMN/FDP-m=0.850/0.280/0,316),

### (Repolymerization)

To 68.70 g of the obtained depolymerization sample were newly added insufficient monomer components as shown in Table 2 in order to reach the composition ratio of the Polymer G (additional amount in Table 2), and a repolymerization product of the polymer was produced in accordance with Example 6 of Japanese Patent Application Laid-Open Publication No. 2013-064118.

### Example 9

To 20.00 g of the depolymerization sample obtained in Example 8 were newly added insufficient monomer components as shown in Table 2 in order to reach the composition ratio of the Polymer G (additional amount in Table 2), and a repolymerization product of the polymer was produced in accordance with Example 6 of Japanese Patent Application Laid-Open Publication No. 2013-064118.

### Example 10

To 10.00 g of the depolymerization sample obtained in Example 8 were newly added insufficient monomer components as shown in Table 2 in order to reach the composition ratio of the Polymer G (additional amount in Table 2), and a repolymerization product of the polymer was produced in accordance with Example 6 of Japanese Patent Application Laid-Open Publication No. 2013-064118.

### Example 11

To 5.00 g of the depolymerization sample obtained in Example 8 were newly added insufficient monomer components as shown in Table 2 in order to reach the composition ratio of the Polymer G (additional amount in Table 2), and a repolymerization product of the polymer was produced in accordance with Example 6 of Japanese Patent Application Laid-Open Publication No. 2013-064118.

### Example 12

### (Depolymerization of Polymer G)

To 173.05 g of the pellet Polymer G (the total amount of monomers incorporated in the Polymer G corresponds to about 0.5 mol) were added 315.28 g of dimethyl carbonate, 3.461 g of NaOMe, and 31.149 g of methanol, and the mixture underwent a reaction at 60° C. for 2 hours. To the resulting reaction mixture were added 125 g of water, 17 g of a 10% by mass HCl aqueous solution, and 300 g of toluene, and the mixture was stirred at 60° C. Thereafter, the resulting aqueous phase was removed. An operation of washing the residual organic phase with water was conducted until the resulting aqueous phase had a conductivity of not more than 10 μS/cm, and then the resulting organic phase was filtered to remove insoluble matter. The organic phase was concentrated to obtain a crude product. To the crude product were added 288 g of methanol and 25.2 g of potassium hydroxide, and the mixture underwent a reaction at a room temperature for 2 hours. To the reaction mixture were added 50 g of a 10% by mass HCl aqueous solution and 150 g of water, and the mixture was stirred at a room temperature for one hour. The stirred mixture was suction-filtered, and the residue was washed with warm water until the filtrate had a conductivity of not more than 10 μS/cm. Thereafter, the washed residue was dried under a reduced pressure at 50° C. to obtain 140 g of a white solid matter (calculation of molar ratio from HPLC measurement: BPEF/DMN/FDP-m=0.850/0.248/0.306).

### (Repolymerization)

To 69,81 g of the obtained depolymerization sample were newly added insufficient monomer components as shown in Table 3 in order to reach the composition ratio of the Polymer G (additional amount in Table 3), and a repolymerization product of the polymer was produced in accordance with Example 6 of Japanese Patent Application Laid-Open Publication No. 2013-064118.

### Example 13

To 20.00 g of the depolymerization sample obtained in Example 12 were newly added insufficient monomer components as shown in Table 3 in order to reach the composition ratio of the Polymer G (additional amount in Table 3), and a repolymerization product of the polymer was produced in accordance with Example 6 of Japanese Patent Application Laid-Open Publication No. 2013-064118.

### Example 14

To 10.00 g of the depolymerization sample obtained in Example 12 were newly added insufficient monomer components as shown in Table 3 in order to reach the composition ratio of the Polymer G (additional amount in Table 3), and a repolymerization product of the polymer was produced in accordance with Example 6 of Japanese Patent Application Laid-Open Publication No. 2013-064118.

### Example 15

To 5.00 g of the depolymerization sample obtained in Example 12 were newly added insufficient monomer components as shown in Table 3 in order to reach the composition ratio of the Polymer G (additional amount in Table 3), and a repolymerization product of the polymer was produced in accordance with Example 6 of Japanese Patent Application Laid-Open Publication No. 2013-064118.

### Example 16

### (Depolymerization of Polymer G)

To 242.27 g of the pellet Polymer G (the total amount of monomers incorporated in the Polymer G corresponds to about 0.7 mol) were added 441.39 g of dimethyl carbonate and 24.227 g of a methanol solution containing LiOMe at a concentration of 10% by mass, and the mixture underwent a reaction at 60° C. for 2 hours. To the resulting reaction mixture were added 63 g of water and 24 g of a 10% by mass HCl aqueous solution, and the mixture was stirred at 65° C. Thereafter, the resulting organic phase was filtered to remove insoluble matter. The organic phase was concentrated to obtain a crude product. To the crude product were added 359 g of methanol and 39.3 g of potassium hydroxide, and the mixture underwent a reaction at a room temperature for 2 hours. To the reaction mixture were added 105 g of a 10% by mass HCl aqueous solution and 280 g of water, and the mixture was stirred at a room temperature for one hour. The stirred mixture was suction-filtered, and the residue was washed with water and then was dried under a reduced pressure at 50° C. to obtain 238 g of a white solid matter (calculation of molar ratio from HPLC measurement: BPEF/DMN/FDP-m=0.850/0.382/0.486).

### (Repolymerization)

To 20,00 g of the obtained depolymerization sample were newly added insufficient monomer components as shown in Table 4 in order to reach the composition ratio of the Polymer G (additional amount in Table 4), and a repolymerization product of the polymer was produced in accordance with Example 6 of Japanese Patent Application Laid-Open Publication No. 2013-064118.

### Example 17

To 10.00 g of the depolymerization sample obtained in Example 16 were newly added insufficient monomer components as shown in Table 4 in order to reach the composition ratio of the Polymer G (additional amount in Table 4), and a repolymerization product of the polymer was produced in accordance with Example 6 of Japanese Patent Application Laid-Open Publication No. 2013-064118.

### Example 18

To 5.00 g of the depolymerization sample obtained in Example 16 were newly added insufficient monomer components as shown in Table 4 in order to reach the composition ratio of the Polymer G (additional amount in Table 4), and a repolymerization product of the polymer was produced in accordance with Example 6 of Japanese Patent Application Laid-Open Publication No. 2013-064118.

Tables 2 to 4 show the evaluation results of Examples 8 to 18. Table 5 shows the evaluation results of the Polymer G as a blank.

TABLE 2

| | | | | | | Analysis results | | | | |
|---|---|---|---|---|---|---|---|---|---|---|
| | | Composition ratio or feed amount | | | | | Molecular | Refractive | | |
| Composition ratio before | | BPEF | EG | DMN | FDP-m | Tg | weight | index | Abbe's | Birefringence |
| depolymerization (mol) | | 0.85 | 0.15 | 0.30 | 0.70 | [° C.] | Mw | nD | number | [×10⁻⁴] |
| Example 8 | Depolymerization | 68.70 | | | | 133.9 | 44200 | 1.6427 | 22.4 | 1.45 |
| | product (g) | | | | | | | | | |
| | Additional amount (g) | 0 | 17.00 | 1.29 | 16.98 | | | | | |
| Example 9 | Depolymerization | 20.00 | | | | 134.1 | 46700 | 1.6427 | 22.4 | 1.04 |
| | product (g) | | | | | | | | | |
| | Additional amount (g) | 33.65 | 17.00 | 6.99 | 26.33 | | | | | |
| Example 10 | Depolymerization | 10.00 | | | | 134.3 | 42300 | 1.6427 | 22.4 | 0.60 |
| | product (g) | | | | | | | | | |
| | Additional amount (g) | 40.56 | 17.00 | 8.16 | 28.25 | | | | | |
| Example 11 | Depolymerization | 5.00 | | | | 133.8 | 42100 | 1.6426 | 22.2 | 0.59 |
| | product (g) | | | | | | | | | |
| | Additional amount (g) | 44.02 | 17.00 | 8.75 | 29.21 | | | | | |

TABLE 3

| | | | | | | Analysis results | | | | |
|---|---|---|---|---|---|---|---|---|---|---|
| | | Composition ratio or feed amount | | | | | Molecular | Refractive | | |
| Composition ratio before | | BPEF | EG | DMN | FDP-m | Tg | weight | index | Abbe's | Birefringence |
| depolymerization (mol) | | 0.85 | 0.15 | 0.30 | 0.70 | [° C.] | Mw | nD | number | [×10⁻⁴] |
| Example 12 | Depolymerization | 69.81 | | | | 133.9 | 40500 | 1.6429 | 22.5 | −0.52 |
| | product (g) | | | | | | | | | |
| | Additional amount (g) | 0.00 | 17.00 | 0.60 | 16.56 | | | | | |
| Example 13 | Depolymerization | 20.00 | | | | 134.3 | 45400 | 1.6428 | 22.5 | 0.50 |
| | product (g) | | | | | | | | | |
| | Additional amount (g) | 33.87 | 17.00 | 6.83 | 26.27 | | | | | |
| Example 14 | Depolymerization | 10.00 | | | | 134.2 | 43200 | 1.6430 | 22.4 | 0.63 |
| | product (g) | | | | | | | | | |
| | Additional amount (g) | 40.67 | 17.00 | 8.08 | 28.22 | | | | | |
| Example 15 | Depolymerization | 5.00 | | | | 134.1 | 45100 | 1.6428 | 22.4 | 0.76 |
| | product (g) | | | | | | | | | |
| | Additional amount (g) | 44.07 | 17.00 | 8.71 | 29.20 | | | | | |

TABLE 4

| | | | | | | Analysis results | | | | |
|---|---|---|---|---|---|---|---|---|---|---|
| | | Composition ratio or feed amount | | | | | Molecular | Refractive | | |
| Composition ratio before | | BPEF | EG | DMN | FDP-m | Tg | weight | index | Abbe's | Birefringence |
| depolymerization (mol) | | 0.85 | 0.15 | 0.30 | 0.70 | [° C.] | Mw | nD | number | [×10⁻⁴] |
| Example 16 | Depolymerization | 20.00 | | | | 135.1 | 42800 | 1.643 | 21.9 | −2.31 |
| | product (g) | | | | | | | | | |
| | Additional amount (g) | 35.65 | 17.00 | 6.37 | 24.95 | | | | | |
| Example 17 | Depolymerization | 10.00 | | | | 135.1 | 42500 | 1.643 | 22.5 | −1.15 |
| | product (g) | | | | | | | | | |
| | Additional amount (g) | 41.56 | 17.00 | 7.85 | 27.56 | | | | | |
| Example 18 | Depolymerization | 5.00 | | | | 134.1 | 43000 | 1.643 | 22.5 | −0.37 |
| | product (g) | | | | | | | | | |
| | Additional amount (g) | 44.52 | 17.00 | 8.59 | 28.87 | | | | | |

TABLE 5

| | | | | | Analysis results | | | | |
|---|---|---|---|---|---|---|---|---|---|
| | Composition ratio or feed amount | | | | | Molecular | Refractive | | |
| Composition | BPEF | EG | DMN | FDP-m | Tg | weight | index | Abbe's | Birefringence |
| ratio (mol) | 0.85 | 0.15 | 0.30 | 0.70 | [° C.] | Mw | nD | number | [×10⁻⁴] |
| Blank (g) | 47.47 | 17.00 | 9.33 | 30.17 | 132 | 35000 | 1.642 | 22.5 | 1 |

As apparent from the results shown in Tables 2 to 5, suitable supplementation of insufficient monomeric raw materials to the decomposition product obtained by the depolymerization step increased the molecular weight of the polymer in repolymerization and allowed the obtained polymer to have excellent optical characteristics.

### Comparative Example 1

### (Depolymerization of Polymer G)

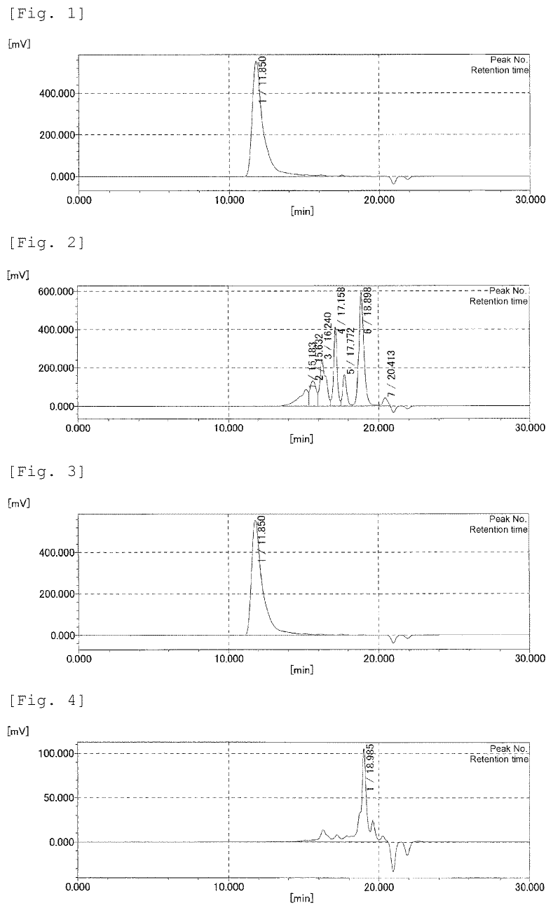

In accordance with the methods described in Patent Documents 1 and 2, to the pellet Polymer G obtained in Example 8 was added EG, and the mixture was heated at 200 to 250° C. for about 3 hours to depolymerize the Polyester F. The molecular weight distribution of the Polymer G before depolymerization is shown in FIG. 1, and the molecular weight distribution of the decomposition product obtained by depolymerization is shown in FIG. 2. The comparison between FIG. 1 and FIG. 2 apparently shows that some kind of decomposition product obtained by depolymerization has a wide molecular weight distribution. This revealed that the Polymer G was not decomposed into monomer units.

That is, it was proved that the conventional method including excessive addition of the solvent (ethylene glycol) and heating at a high temperature allowed depolymerization of a widely used polyester such as PET to proceed while the conventional method did not allow depolymerization of the fluorene skeleton-containing polymer,

### Comparative Example 2

### (Depolymerization of Polymer G)

In accordance with the method described in Patent Document 3, to 10 g of the pellet Polymer G obtained in Example 8 was added 20 g of water, and the mixture was treated with subcritical water at 300° C. under 8 MPa nitrogen atmosphere for 2 hours to depolymerize the Polyester G. The molecular weight distribution of the Polymer G before depolymerization is shown in FIG. 3, and the molecular weight distribution of the decomposition product obtained by depolymerization is shown in FIG. 4. The comparison between FIG. 3 and FIG. 4 apparently shows that some kind of decomposition product obtained by depolymerization has a wide molecular weight distribution. This revealed that the Polymer G was not decomposed into monomer units.

That is, it was proved that the conventional method including hydrolysis with subcritical water (hydrolysis method) allowed depolymerization of a widely used polyester such as PET to proceed while the conventional method did not allow depolymerization of the fluorene skeleton-containing polymer.

Also from the results of Comparative Examples 1 and 2, it is understood that the conventional method is difficult in depolymerizing the fluorene skeleton-containing polyester-series resin differently from the widely used polyester such as PET.

### Example 19

### (Preparation of Polymer H)

In accordance with a conventional method, Polymer H having a carbonate ester bond was prepared in which a constitutional unit derived from BPEF was 100% by mol in a diol unit.

### (First Decomposition Step)

To 46.618 g of the Polymer H (the total amount of monomers incorporated in the Polymer corresponds to about 0.1 mol) were added 22.52 g of dimethyl carbonate, 0.9324 g of NaOMe, and 8.3912 g of methanol, and the mixture underwent a reaction at 65° C. for one hour. To the resulting reaction mixture were added 60 g of toluene, 40 g of water, and 4.6 g of a 10% by mass HCl aqueous solution, and the mixture was stirred at 75° C. for 15 minutes and then subjected to liquid-liquid separation, the separated organic phase was washed with 40 g of water, and this water-washing step was repeated three times. Then, the washed product was filtered to remove insoluble matter,

### (Second Decomposition Step)

The organic phase was concentrated to obtain a crude product (a first decomposition product), To the crude product were added 96 g of methanol and 4.49 g of potassium hydroxide, and the mixture underwent a reaction for one hour at a room temperature. To the reaction mixture were added 8 g of a 108 by mass HCL aqueous solution and 30 g of water, and the resulting mixture was stirred at 5 to 10° C. for one hour. Thereafter, the mixture was filtered to obtain 55 g of crude BPEF crystal.

### (Purification Step of BPEF)

To the above-described crude crystal was added 165 g of toluene, and the mixture was completely dissolved at 75° C., Thereafter, the mixture was allowed to slowly cool to a room temperature and was stirred at 5 to 10° C. for one hour. The filtration was conducted, and the residue was dried under a reduced pressure at 80° C. for 12 hours to obtain 22.9 g of a white solid matter containing BPEF (yield 52%, LC purity 98.3%).

For Example 19, Table 6 shows the composition of the first decomposition product, and the yield and purity of the diol (BPEF) after the purification step of BPEF.

TABLE 6

| | First decomposition step | | | | |
|---|---|---|---|---|---|
| | product LC-MS composition | | | Diol | |
| | ratio (% by area) | | | Yield | LC purity |
| | Monocarbonate | Dicarbonate | | (% by | (% by |
| | ester form | ester form | Diol | mol) | area) |
| Example 19 | 39.07 | 12.41 | 36.57 | 52 | 98.3 |

As apparent from the results of Table 6, Example 19 produced novel monocarbonate ester form and dicarbonate ester in the first decomposition step and further enabled recovery of the diol through the second decomposition step and the purification step.

### Example 20

### (Preparation of Polymer I)

In accordance with a conventional method, Polymer I having a carbonate ester bond was prepared in which a constitutional unit derived from BPEF was 50% by mol and a constitutional unit derived from BINOL-2EO was 50% by mol in a diol unit.

### (First Decomposition Step)

To 20.324 g of the Polymer I (the total amount of monomers incorporated in the Polymer corresponds to about 0.05 mol) were added 11.26 g of dimethyl carbonate, 0.4065 g of NaOMe, and 3.6583 g of methanol, and the mixture underwent a reaction at 65° C. for 2 hours. To the resulting reaction mixture were added 35 g of toluene, 20 g of water, and 2.3 g of a 10% by mass HCl aqueous solution, and the mixture was stirred at 75° C. for 15 minutes and then subjected to liquid-liquid separation, the separated organic phase was washed with 20 g of water, and this water-washing step was repeated three times. Then, the washed product was filtered to remove insoluble matter,

### (Second Decomposition Step)

The organic phase was concentrated to obtain a crude product (a first decomposition product). To the crude product were added 48 g of methanol and 2.24 g of potassium hydroxide, and the mixture underwent a reaction for one hour at a room temperature. To the reaction mixture were added 4 g of a 10% by mass HCl aqueous solution and 15 g of water, and the resulting mixture was stirred at 5 to 10° C. for one hour. Thereafter, the mixture was filtered, and the filtration residue was dried under a reduced pressure at 50° C. to obtain 17.4 g of a white solid matter (a mixture of BPEF and BINOL-2E0) (LC purity 94.7% and yield 85% (purity and yield as the mixture)).

### Example 21

### (Preparation of Polymer J)

In accordance with a conventional method, Polymer J having a carbonate ester bond was prepared in which a constitutional unit derived from BOPPEF was 50% by mol and a constitutional unit derived from BINOL-2EO was 50% by mol in a diol unit.

### (First Decomposition Step)

To 24,129 g of the Polymer J (the total amount of monomers incorporated in the Polymer corresponds to about 0.05 mol) were added 11.26 g of dimethyl carbonate, 0.4826 g of NaOMe, and 4.324 g of methanol, and the mixture underwent a reaction at 65° C. for 2 hours. To the resulting reaction mixture were added 35 g of toluene, 20 g of water, and 2.3 g of a 10% by mass HCl aqueous solution, and the mixture was stirred at 75° C. for 15 minutes and then subjected to liquid-liquid separation, the separated organic phase was washed with 20 g of water, and this water-washing step was repeated three times. Then, the washed product was filtered to remove insoluble matter,

### (Second Decomposition Step)

The organic phase was concentrated to obtain a crude product. To the crude product were added 48 g of methanol and 2.24 g of potassium hydroxide, and the mixture underwent a reaction for one hour at a room temperature. To the reaction mixture were added 4 g of a 10% by mass HCl aqueous solution and 15 g of water, and the resulting mixture was stirred at 5 to 10° C. for one hour. Thereafter, the mixture was filtered, and the filtration residue was dried under a reduced pressure at 50° C. to obtain 21,7 g of a white solid matter (a mixture of BOPPEF and BINOL-2E0) (LC purity 98.2% and yield 90% (purity and yield as the mixture)).

### INDUSTRIAL APPLICABILITY

The dicarboxylic acid component and/or the diol component obtained by the depolymerization method of the present disclosure has a fluorene skeleton and has excellent heat resistance or optical characteristics, and thus is used as a raw material or an additive of a polyester-series resin or a polycarbonate ester-series resin in various fields including an optical field. Further, the novel carbonate ester form of the diol obtained in the process of the depolymerization method of the present disclosure is used as a raw material for a polycarbonate ester-series resin, a reaction conditioner, and an additive such as a resin additive.

1. A depolymerization method for depolymerizing a fluorene-containing resin, the fluorene-containing resin having a fluorene skeleton and an ester bond and/or a carbonate ester bond, the method comprising allowing the fluorene-containing resin to react with a carbonate ester in the presence of a hydrolysis catalyst to obtain a decomposition product.

2. The depolymerization method according to claim 1, wherein the decomposition product contains a monocarbonate ester form of a diol and/or a dicarbonate ester form of a diol.

3. The depolymerization method according to claim 2, wherein the decomposition product further contains a dicarboxylic acid and/or an ester thereof.

4. The depolymerization method according to claim 1, which further comprising allowing the decomposition product to react with an alcohol to obtain a diol.

5. The depolymerization method according to claim 1, wherein the fluorene-containing resin is a polyester-series resin containing, as a polymerization component,
a dicarboxylic acid component represented by the following formula (1): 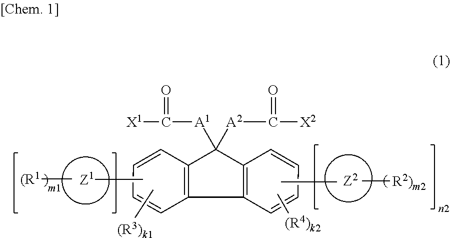
wherein
a ring Z¹ and a ring Z² independently represent an arene ring,
R¹ and R² independently represent a substituent, m1 and m2 independently denote an integer of not less than 0,
n1 and n2 independently denote an integer of 0 to 4,
A¹ and A² independently represent an alkylene group,
X¹ and X² independently represent a hydroxyl group, an alkoxy group, or a halogen atom,
R³ and R⁴ independently represent a substituent, and k1 and k2 independently denote an integer of 0 to 4 and/or
a diol represented by the following formula (2): 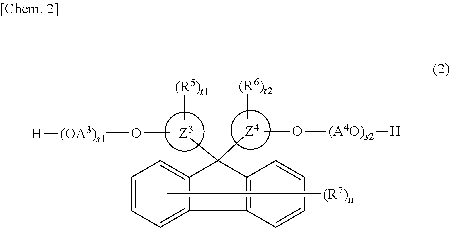
wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

6. The depolymerization method according to claim 5, wherein the fluorene-containing resin is a polyester-series resin containing the dicarboxylic acid component represented by the formula (1) as the polymerization component, and in the formula (1), n1 and n2 each denote 0, or n1 and n2 each denote 1 and the ring Z¹ and the ring Z² independently represent a condensed polycyclic arene ring.

7. The depolymerization method according to claim 5, wherein the fluorene-containing resin is a polyester-series resin containing the diol represented by the formula (2) as the polymerization component.

8. The depolymerization method according to claim 1, wherein the fluorene-containing resin is a polycarbonate ester-series resin containing, as a polymerization component, a diol represented by the following formula (2): 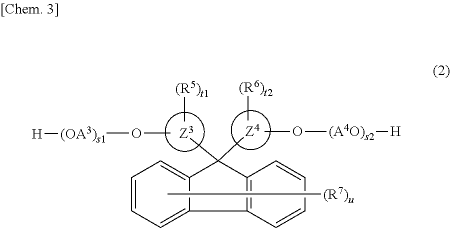
wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer an of 0 to 8.

9. The depolymerization method according to claim 1, wherein the fluorene-containing resin contains, as a polymerization component, a diol represented by the following formula (5): 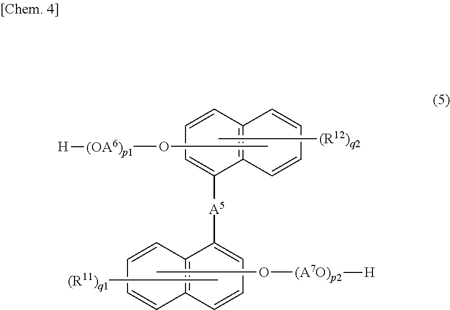
wherein
A⁵ represents a direct bond (a single bond) or an alkylene group,
A⁶ and A⁷ independently represent an alkylene group, p1 and p2 independently denote an integer of not less than 0,
R¹¹ and R¹² independently represent a substituent, and q1 and q2 independently denote an integer of 0 to 6.

10-11. (canceled)

12. A method for recovering a fluorene skeleton-containing monomer component, the method comprising
a first decomposition step of allowing a polyester-series resin with a fluorene skeleton and an ester bond to react with a carbonate ester in the presence of a hydrolysis catalyst to decompose the polyester-series resin, obtaining a decomposition product containing a dicarboxylic acid and/or an ester thereof, and a monocarbonate ester form of a diol and/or a dicarbonate ester form of a diol, and
a second decomposition step of allowing the decomposition product to react with an alcohol to obtain a diol.

13. A method for recovering a fluorene skeleton-containing monomer component, the method comprising
a first decomposition step of allowing a polycarbonate ester-series resin with a fluorene skeleton and a carbonate ester bond to react with a carbonate ester in the presence of a hydrolysis catalyst to decompose the polycarbonate ester-series resin, obtaining a first decomposition product containing a monocarbonate ester form of a diol and/or a dicarbonate ester form of a diol, and
a second decomposition step of allowing the first decomposition product to react with an alcohol to obtain a diol.

14. A monocarbonate ester form of a diol represented by the following formula (3): 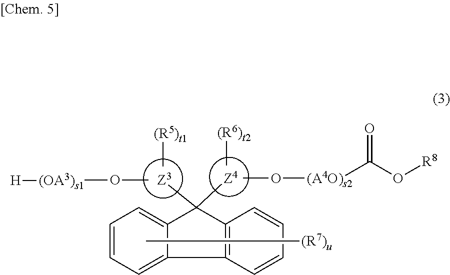
wherein
a ring Z³ and a ring Z⁴ independently represent an arene ring,
A³ and A⁴ independently represent an alkylene group, s1 and s2 independently denote an integer of not less than 0,
R⁸ represents a hydrocarbon group,
R⁵ and R⁶ independently represent a substituent, t1 and t2 independently denote an integer of not less than 0,
R⁷ represents a substituent, and u denotes an integer of 0 to 8.

15. A dicarbonate ester form of a diol represented by the following formula (4): 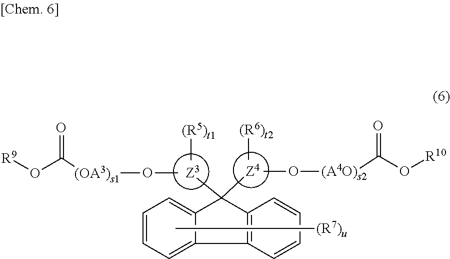
wherein
a ring Z³ and a ring Z⁴ independently represent an arene ring,
A³ and A⁴ independently represent an alkylene group, s1 and s2 independently denote an integer of not less than 0,
R⁹ and R¹⁰ independently represent a hydrocarbon group,
R⁵ and R⁶ independently represent a substituent, t1 and t2 independently denote an integer of not less than 0,
R⁷ represents a substituent, and u denotes an integer of 0 to 8.

16. A process for producing a monocarbonate ester form of a diol recited in claim 14, the process comprising allowing a fluorene-containing resin with a fluorene skeleton and an ester bond and/or a carbonate ester bond to react with a carbonate ester in the presence of a hydrolysis catalyst to decompose the fluorene-containing resin, wherein
the fluorene-containing resin is a fluorene-containing resin containing, as a polymerization component, a diol represented by the following formula (2): 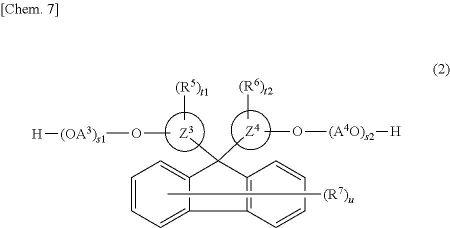
wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

17. A process for producing a dicarbonate ester form of a diol recited in claim 15, the process comprising allowing a fluorene-containing resin with a fluorene skeleton and an ester bond and/or a carbonate ester bond to react with a carbonate ester in the presence of a hydrolysis catalyst to decompose the fluorene-containing resin, wherein
the fluorene-containing resin is a fluorene-containing resin containing, as a polymerization component, a diol represented by the following formula (2): 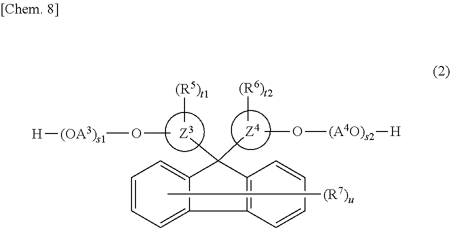
wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

18. A method for recycling a fluorene-containing resin with a fluorene skeleton and an ester bond and/or a carbonate ester bond, the method comprising
a depolymerization step of allowing the fluorene-containing resin to react with a carbonate ester in the presence of a hydrolysis catalyst to obtain a decomposition product, and
a polymerization step of polymerizing the decomposition product obtained in the depolymerization step to obtain a newly produced fluorene-containing resin.

19. The recycling method according to claim 18, wherein a monomeric raw material for the newly produced fluorene-containing resin is supplemented in the polymerization step.

20. The depolymerization method according to claim 2, which further comprising allowing the decomposition product to react with an alcohol to obtain a diol.

21. The depolymerization method according to claim 2, wherein the fluorene-containing resin is a polyester-series resin containing, as a polymerization component,
a dicarboxylic acid component represented by the following formula (1): 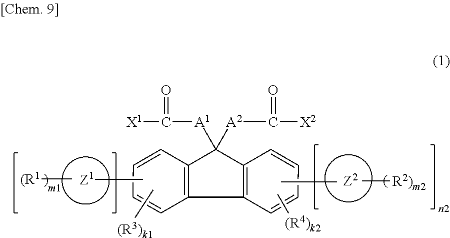
wherein
a ring Z¹ and a ring Z² independently represent an arene ring,
R¹ and R² independently represent a substituent, m1 and m2 independently denote an integer of not less than 0,
n1 and n2 independently denote an integer of 0 to 4,
A¹ and A² independently represent an alkylene group,
X¹ and X² independently represent a hydroxyl group, an alkoxy group, or a halogen atom,
R³ and R⁴ independently represent a substituent, and k1 and k2 independently denote an integer of 0 to 4 and/or
a diol represented by the following formula (2): 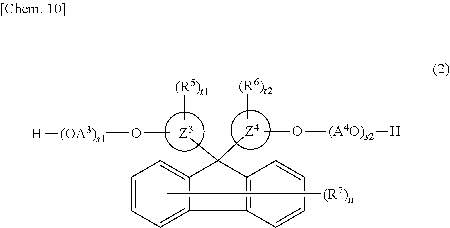
wherein
a ring Z³ and a ring Z⁴ independently represent an arene ring,
A³ and A⁴ independently represent an alkylene group, s1 and s2 independently denote an integer of not less than 0,
R⁵ and R⁶ independently represent a substituent, t1 and t2 independently denote an integer of not less than 0,
R⁷ represents a substituent, and u denotes an integer of 0 to 8.

22. The depolymerization method according to claim 2, wherein the fluorene-containing resin contains, as a polymerization component, a diol represented by the following formula (5): 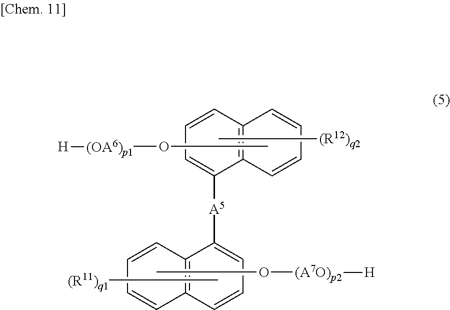
wherein
A⁵ represents a direct bond (a single bond) or an alkylene group,
A⁶ and A⁷ independently represent an alkylene group, p1 and p2 independently denote an integer of not less than 0,
R¹¹ and R¹² independently represent a substituent, and q1 and q2 independently denote an integer of 0 to 6.
